(19) United States
(12) Patent Application Publication
(10) Pub. No.: US 2018/0128811 A1
(43) Pub. Date: May 10, 2018

(54) ASPIRIN RESPONSE AND REACTIVITY TEST AND ASPIRIN COMPLIANCE TEST USING SYNTHETIC COLLAGEN

(71) Applicant: JNC Corporation, Tokyo (JP)

(72) Inventor: William M. TROLIO, Horsham, PA (US)

(73) Assignee: JNC Corporation

(21) Appl. No.: 14/412,994

(22) PCT Filed: Jul. 5, 2013
(86) PCT No.: PCT/US2013/049418

§ 371 Date: Jan. 5, 2015

Related U.S. Application Data
(60) Provisional application No. 61/668,820, filed on Jul. 6, 2012.
(60) Provisional application No. 61/680,111, filed on Aug. 6, 2012.
(60) Provisional application No. 61/681,485, filed on Aug. 9, 2012.

(51) Int. Cl. G01N 33/50

(52) U.S. Cl. CPC G01N 33/502; G01N 2800/52; G01N 33/5044

(57) ABSTRACT

The present invention provides platelet aggregation assays using synthetic collagen, methods of measuring a donor's platelet aspirin sensitivity status and residual platelet reactivity, and aspiring therapy compliance using synthetic collagen. The invention further provides kits useful in the assays and methods.

This application claims priority to U.S. Provisional Application 61/668,820 filed on Jul. 6, 2012, which is incorporated herein in its entirety.

### BACKGROUND OF THE INVENTION

The conventional, primary need for an effective assessment of platelet response and reactivity is in the field of cardiology. The public health incidence and burden of heart attack, stroke and related cardiovascular and thrombotic diseases are well known. The medical community has long recommended the use of aspirin in primary care to reduce cardiovascular risk. Aspirin (salicylate based compounds) ingestion inhibits the COX 1 pathway and modifies COX 2 enzymatic processes, which then precludes all subsequent events necessary for platelet aggregation. Since the ingestion of aspirin can inhibit platelet aggregation, it has been given as a therapy to prevent undesired platelet aggregation, which can be a source of many heart attacks, strokes or other thrombotic events.

In addition to cardiology, other medical specialties have reported the benefits of aspirin for their patients including anesthesiology; diabetology; nephrology; neurology, oncology; orthopedics; and rheumatology.

Despite the benefits of aspirin therapy in many individuals, aspirin therapy is not effective in some individuals as it does not cause the desired inhibition of platelet aggregation or its effect is shorter than the dosing interval (some patients may only get 6 to 12 hours of protection rather than 24 hours resulting in an above baseline risk for the patient in the time between doses). In other individuals, aspirin therapy can be harmful as it creates an increased risk of unwanted bleeding complications because the aspirin seems to block platelet activity altogether so that the blood does not clot when physiologically necessary.

Thus, there is a need for a reliable tool to assess and manage aspirin's response and reactivity on platelet aggregation as well as assess patient compliance. Traditionally, a patient's response to aspirin is tested by testing platelet activity in the presence of aspirin with a platelet aggregation test. The “gold standard” of platelet aggregation tests is light transmission aggregometry (LTA), which utilizes collagen from biological sources as the agonist to bring about platelet aggregation, as a measure of the degree or extent of platelet response or inhibition to aggregation. However, there are multiple analytical, performance and interpretive issues as well as the risk of infectious disease transmission when using biological material. Biologically derived products, whether ‘natural,’ processed, manufactured by fermentation, cell culture or similar processes, or recombinant, all share the following drawbacks: carry a risk of infectious disease transmission; have lot to lot variability (regarding the ratio of active materials, performance, chemical characteristics, solubility, stability, moisture content, and process contaminants); differing bio-profiles depending upon the location the product was made; differences caused by processing; and environment, geographic and dietary differences affecting the source animal or culture. In addition, biologically derived collagen does not behave in the stoichiometric manner typical of chemical analytics. For example, it does not dilute, does not have a quantitative relationship to the analyte, and maybe insensitive to the (aspirin) analyte, etc.

There are two agonists (compounds that will normally cause platelets to aggregate) that are routinely used to detect aspirin's effect on platelet function as measured by light transmission aggregometry assays (LTA): Arachidonic Acid (used to evaluate the inhibitory effects of aspirin) and collagen (used to evaluate platelet activation, heritable platelet dysfunctions and inhibitory effects of aspirin).

In addition to the LTA there are many other platelet aggregation tests commercially available. However, they all have many short-comings, the common thread linking the currently available assays is that they do not provide the clinician with information that changes patient outcome. Many of these tests are described below.

Commercially Available Tests for Aspirin Effect on Platelet Function

One commercially available test is sold by Accumetrics® and is called the VerifyNow® Aspirin Test. This test utilizes a whole blood system for platelet function testing. It is intended as a qualitative test to aid in the detection of platelet dysfunction due to aspirin ingestion in citrated whole blood for the point of care or laboratory setting. Reported limitations for this test include the following observations. The test shows platelet inhibition in the absence of aspirin. A GRAVITAS trial result showed that the test had no predictive value for antiplatelet therapy. This test cannot be used in patients with heritable platelet defects. U.S. Pat. Nos. 7,595,169; 7,205,115; 6,016,712; 5,922,551; and D409,758 are reported to relate to this test.

Another commercially available test also sold by Accumetrics® is the VerifyNow® P2Y12 Test. This test is an assay designed to assess platelet function based on the ability of activated platelets to bind fibrinogen. It is intended as a whole blood test used in the laboratory or point of care setting to measure the level of platelet P2Y12 receptor blockade. Reported limitations of this test include the following observations. This test shows platelet inhibition in 30% of patients in the absence of aspirin and cannot be used in patients with heritable platelet defects. The results of the test are affected by IIb/IIIa & phosphodiesterase inhibitors. The P2Y12 test's arbitrary units and percent of platelet inhibition are not equivalent. U.S. Pat. No. 7,790,362 is reported to relate to this test.

Another product available is AspirinWorks® by Spectracell/Corgenix®. This is a competitive ELISA assay performed on diluted urine samples. It is intended as a qualitative test to determine levels of 11-Dehydro Thromboxane B2 (11dhTxB2) in human urine, which aids in the determination of platelet response to aspirin (platelet inhibition—not reactivity). Reported limitations of this test include the following observations. This test is not specific for platelets, and monocytes & macrophages affect the test result. This test requires the use of a non-automated ELISA, which causes the test to be time consuming and cumbersome. In addition, this test also requires a creatinine (blood chemistry) test result to be able to interpret the test result U.S. patents and applications U.S. Pat. Nos. 8,168,400; 8,105,790; 7,727,730; 2003/0133873; and 2003/0124615 are reported to relate to this test.

Another test available is the Platelet Reactivity Test® by Placor®. This test is marketed as global test for platelet reactivity. It is intended as a point-of-care device to measure the platelet reactivity of aspirin and antiplatelet drugs. U.S. Pat. Nos. 7,534,620; and 7,309,607 are reported to relate to this test. PlaCor PRT is a global test of platelet function, like a bleeding time; it shows a modest agreement with comparable tests (r value of 0.60), and results depend considerably on platelet count. Wurtz et al., Thromb Res. 2012 November; 130(5):753-8.

Another test is the ASPITest® by Roche® (Verum Multiplate®). This test is an impedance based analysis of platelet function in whole blood using arachidonic acid. It is intended as a routine platelet aggregation study for the evaluation of normal platelet function. Reported limitations of this test include the following observations. This test has been reported to show platelet inhibition in the absence of aspirin. A large sample size is required and the test uses an anticoagulant not recommended for platelet function tests. The 15 μM concentration is 50% higher than the typical maximum Arachidonic Acid concentration. According to its package insert, ASPITest® is Sensitive towards cyclooxygenase inhibition, GpIIb/IIIa antagonists and a deficiency of GpIIb/IIIa receptors, and not aspirin specifically.

Another test is the Siemens® PFA 100 Col/ADP Test®. This test measures primary hemostasis by determining the time from the start of the test until the platelet plug occludes the aperture, and reports the time interval as the Closure Time (CT). The PFA Collagen/ADP Test Cartridges are utilized for the differentiation of aspirin effect on platelets versus other platelet dysfunctions. It is insensitive to aspirin, yet sensitive to Von Willebrand Disease (VWD), low platelet counts, and other platelet dysfunctions. U.S. patent applications 2007/0254325 and 2007/0254324 are reported to relate to this test.

Another test is the Siemens® PFA 200 P2Y® test. This test provides automated biometric impedance assessment of platelet function. It is intended to detect the P2Y12 receptor blockade in patients undergoing therapy with a P2Y12 receptor blockade antagonist. Reported limitations of this test include the following observations. The specificity has been reported to be less than 42% and the results vary with anticoagulant used for specimen collection. The test is also dependent on von Willebrand factor and hematocrit. U.S. patent applications 2007/0254325 and 2007/0254324 are reported to relate to this test.

Additional tests are discussed in U.S. patent applications 2010/017339; 2011/0045481; 2008/0207681; and 2010/0137161.

Thus, there remains a need for a more reliable platelet activity test that does not use an animal derived collagen as the agonist and that provides the clinician with a residual platelet reactivity result that is actionable. There also remains a need to have test that could be routinely used to test/monitor a patient's compliance with aspirin therapy. The present invention meets this need.

### SUMMARY OF THE INVENTION

The present invention provides assays that determine a donor's aspirin sensitivity status using synthetic collagen. Exemplary assays include the assessment of platelet aggregation using light transmission aggregation assay (LTAA) and Flow Cytometry.

In another embodiment, the present invention provides methods for determining a donor's platelet aspirin sensitivity status (which may be aspirin hypersensitive, average aspirin sensitive, and aspirin non-responsive) and/or determining the degree of aspirin sensitivity, degree of aspirin hypersensitivity or the degree of aspirin non-responsiveness. Assays of the invention can also be used determine if the aspirin dose is adequate for the dosing interval and for the amount prescribed.

Certain embodiments of the present invention utilize synthetic collagen at amounts at least 1000 fold less than similar assays using biological collagen.

Certain embodiments of the present invention involve performing platelet aggregation assays, and assessing platelet aggregation with methods such as, but not limited to, a light transmission aggregation assay (LTAA) and flow cytometry, before a donor ingests aspirin and performing another aggregation assay after the donor ingests aspirin to determine the donor's platelet aspirin response. Certain embodiments use aspirinated plasma instead of having the donor ingest the aspirin.

Certain embodiments involve performing multiple platelet aggregation assays over various dilutions of synthetic collagen to obtain a dilution profile. Analyzing the results of the platelet aggregation assays over the dilution profile is used to determine the donor's platelet sensitivity status as well as residual or remaining platelet functionality or reactivity. Physicians can then utilize the results to assist them in decision making regarding a suitable therapy.

In certain embodiments, the synthetic collagen is a synthetic collagen that has the ability to self-assemble into a triple helix to form fibrils and which mimics human type I collagen. In certain embodiments the synthetic collagen comprises a polypeptide having a peptide fragment represented by the formula (I):

-(Pro-X-Gly)ₙ (I)

wherein X represents Hyp; and n represents an integer of from 20 to 5,000; and wherein the polypeptide has a molecular weight at a range of from 10,000 to 500,000. In certain embodiments, n=20-250.

The present invention also provides kits for testing platelet aggregation, comprising a vial of synthetic collagen at a concentration from about 0.50 ng/mL to about 500.0 ng/mL. In certain embodiments, the vial may contain synthetic collagen at a concentration from about 2.0 ng to about 640 ng. The kit may contain instructions for use of the synthetic collagen in the assay. In some embodiments the kits contain a vial of synthetic collagen at a concentration of about 50 ng/mL, and optionally diluents (s), and positive and/or negative controls.

The present invention also provides kits comprising multiple additional vials of synthetic collagen at different concentrations ranging from about 0.50 ng/mL to about 500.0 ng/mL, or about 2.0 ng/mL to about 640 ng/mL and optionally diluents and positive and/or negative controls. The present invention also provides kits comprising multiple additional vials of synthetic collagen at different concentrations ranging from about 2.5 ng/mL to about 500.0 ng/mL, and optionally diluents and positive and/or negative controls.

In certain embodiments (including the methods described herein and the kits), the synthetic collagen is supplied and/or stored in a polypropylene homomer container. In certain embodiments, the cap is the same material as the vial/tube. In certain embodiments, the container has an additional internal seal or a cap having a secondary seal molded therein. In certain embodiments, the container contains all of the above described characteristics

The present invention also provides a method of testing patient compliance, recently identified as a significant unmet medical need, that the invention meets for with-aspirin therapy using synthetic collagen in platelet aggregation assays.

### BRIEF DESCRIPTION OF THE FIGURES

FIG. 1A shows an LTAA run with synthetic collagen. Different amounts of synthetic collagen were used (amounts provided under the column entitled “Details”). The “in-test” concentrations (the amount of collagen used in each LTAA) of synthetic collagen ranged from 2.5 ng/mL to 25 ng/mL. FIG. 1A is provided to show that the columns of data labeled PA, PS, SA, SS, LP, DA, MA and FA are measured parameters. The remaining parameter, AUC, is calculated, and of these, PA is considered a primary measurement. FIG. 1A shows that synthetic collagen does work over an extended range of dilutions with aspirinated plasma, which is useful to know for developing control and calibration plasmas, which may be used to set parameters for the various assay measurements and to assure proper functioning of the entire assay system. FIG. 1A also shows that that over this range of concentrations, the results would all be interpreted as normal collagen responses despite the presence of aspirin—which potentially could be a clinically dangerous interpretation if the presence of aspirin was not noted on the report to the ordering physician. There is no tell-tale sign of aspirin presence in these results. From this figure it appears that channels 3 and 4 results are the least sensitive to aspirin. Channels/samples 1-4 plasma samples were aspirinated with 25 μl of an aspirin solution added to the platelets and samples 5-8 had 5 μl of an aspirin added to platelets. To prepare the aspirin solution, a 500 mg aspirin tablet was crushed and suspended in a diluent, and the resulting solution had about 150 mg/mL of aspirin.

FIG. 1b shows how to basically calculate the slope in a readout of raw data from an LTAA. This figure shows the 0% baseline reversed with the 100% aggregation on the other figures in the application.

FIG. 2 shows the normal or average donor response when using biological collagen in an LTAA.

FIG. 3 shows the typical response to biological collagen from a normal/average aspirin responder who has ingested aspirin.

FIG. 4 shows responses to various dilutions of synthetic collagen from a normal/average aspirin responder who has ingested aspirin.

FIG. 5 shows LTAA results using biological collagen without aspirin ingestion where the response appears “normal” but where the donor is actually aspirin resistant (aspirin non-responsive).

FIG. 6 shows LTAA results after aspirin ingestion, where the LTAA was run with biological collagen. The donor appears to have a normal/average aspirin response when using the biological collagen.

FIG. 7 shows LTAAs run over a dilution profile of synthetic collagen, showing that the donor is aspirin resistant (aspirin non-responsive).

FIG. 8 shows LTAAs run over multiple dilutions of another biological collagen.

FIG. 9 shows LTAAs run over multiple dilutions of another biological collagen.

FIG. 10 shows slopes from dilution profile LTAAs run using three different donor platelet rich plasma samples (PRP) using synthetic collagen. Four different “in-test” concentrations were used 25.0 ng/mL, 10.0 ng/mL, 5.0 ng/mL and 2.5 ng/mL. One familiar with the responses to synthetic collagen would expect that a normal response (an individual with an average aspirin sensitivity) to have a linear dilution profile (such as seen with donor 7206)—that is, as the concentration goes down, the slope decreases. Donor 7003's response is definitely non-linear, thus indicating that donor 7003 does not have a normal response and thus does not have an average or normal aspirin sensitivity. Biological collagens failed to detect this abnormality.

FIG. 11 provides the results of LTAAs using synthetic collagen for three different donors. The aspirin response status of the three donors is as follows: donor 7003 may be insensitive to aspirin; donor 7225 may be slightly sensitive to aspirin and donor 7206 may show an expected normal response to aspirin.

FIG. 12 provides the results of LTAAs using synthetic collagen. This figure shows the response slope after the donors ingested aspirin and it shows that it corresponds to the slope pattern when the donors had not ingested aspirin (as in FIG. 11). Thus, this figure provides confirmation of what the donor's response was predicted as in FIG. 11 before aspirin was ingested.

FIG. 13 provides the results of LTAAs using synthetic collagen. FIG. 13 shows the “bounce back” in donor 7003, as opposed to a more linear-like slope seen in donor 7225 and 7206.

FIG. 14 shows tests performed using two different biological collagens. The top panel is using collagen from one source “BDC” (a calf skin derived, acid extracted, type 1 collagen) and the bottom panel uses biological collagen from another source (“Chrono Log”)(an equine tendon derived type 1 collagen, with a measurable presence of type III collagen). FIG. 14 shows the comparison of slopes from LTAAs run on biological collagen. Both panels used a platelet donor considered to have a normal or average aspirin sensitivity. Both panels used biological collagen in the LTAs run before (left side—diamonds) and after aspirin was ingested (right side—squares). These results show that the “Chrono Log” biological collagen is totally insensitive to the presence of aspirin. These charts show the two extremes of biological collagen. BDC collagen will detect aspirin, but gives no indication of the degree of responsiveness or resistance. Chrono-Log fails to detect aspirin. Even though the Chrono-Log collagen is a liquid that must be diluted prior to use, further dilutions do not result in it becoming sensitive to the presence of aspirin.

FIG. 15 shows that synthetic collagen and biological collagen generates very similar looking curves, but the synthetic collagen is used at a fraction of the concentration of biological collagen. The AUC values for synthetic collagen (−400) are much higher than for biological collagen (−300), which shows that synthetic collagen provides more sensitive readings and further that synthetic collagen is actually acting differently than biological collagen in the assay.

FIG. 16 shows the results of a collagen-Induced Platelet Aggregation study (see example 1).

FIG. 17 shows the activation of platelets in citrated whole blood by various collagen reagents, including synthetic collagen (see example 1) as assesed with flow cytometery.

FIG. 18 shows the activation of platelets in citrated whole blood by various collagen reagents (see example 1) as assessed with flow cytometry.

FIG. 19 shows the results of an aggregation study, as assessed with flow cytometery, where Aspisol® (an aspirin solution formulated for in vivo use) was supplemented to whole blood prior to activation at concentrations of 1, 5 and 10 μg/ml (see example 1).

FIG. 20 shows the effect of aspirin on collagen-induced platelet activation (see example 1) as assessed with flow cytometry. Aspisol was supplemented to whole blood prior to activation at concentrations of 1, 5 and 10 μg/ml. (see example 1).

### DETAILED DESCRIPTION OF THE INVENTION

The present invention provides assays that determine a donor's aspirin sensitivity status using synthetic collagen. Exemplary assays include, but are not limited to, assessing platelet aggregation using light transmission aggregation assays (LTAA) and whole blood Flow Cytometry. The present invention provides a method for determining a donor's platelet aspirin response status, predicting or informing the user of the degree of the donor's sensitivity to aspirin, as well as a method for predicting a donor's sensitivity to aspirin, by testing the ability of the donor's platelets to aggregate before and after the donor has ingested aspirin (or after the plasma sample has been aspirinized).

The present invention also provides a method for testing a patient's compliance with aspirin therapy by monitoring the patient's platelet response over time. Therapy means both taking the aspirin, and taking the aspirin at the proper time to maintain its anti-platelet effect.

Aspirin is a common drug whose active ingredient is acetylsalicylic acid (ASA or ASS). It is a weak acid that is absorbed across the mucosal lining of the stomach and small intestine. After absorption, ASA is (metabolized) hydrolyzed to acetic acid and salicylic acid.

In most individuals, aspirin causes inhibition of platelet aggregation and thus, aspirin is used in many therapies where it is desired to minimize platelet aggregation. These individuals are sometimes referred to as normal or average aspirin sensitive.

However, in some individuals, even after taking aspirin, the platelets will still aggregate, and hence aspirin therapy would not be beneficial or at the very least would not be beneficial alone. These individuals are often referred to as aspirin resistant or aspirin non-responsive, or high on treatment platelet reactivity (platelets are still highly reactive while patient is on therapy). In this situation, it is important for the physician to know if the patient is compliant or non-compliant in their therapy regimen rather than being non-responsive to aspirin.

In yet other individuals, even very small doses of aspirin cause a severe inhibition of platelet aggregation that could lead to serious bleeding issues. These individuals can be called aspirin hypersensitive. For these individuals aspirin therapy may cause more harm than good because of the increased bleeding risk. In other individuals, aspirin administration causes a life threatening anaphylactic reaction. These individuals are said to be aspirin intolerant. Thus, it is very desirable to be able to test a patient/donor for their response to aspirin to see what effects the aspirin will have on the patient's platelet aggregation to determine whether the aspirin therapy will be useful for preventing unwanted platelet aggregation or whether aspirin therapy should be avoided altogether or perhaps instead be used with another drug to enhance the aspirin effect.

Embodiments of the invention can test for platelet aggregation using methods known in the art, including, but not limited to flow cytometery and light transmission aggregometry (LTA).

Flow cytometry uses whole blood and can be used to detect platelet aggregation. See example 1.

Light Transmission Aggregometry (LTA) is an in vitro diagnostic assay that measures multiple optical parameters from changes in light transmission through Platelet Rich Plasma (PRP) following the addition of an agonist (such as, but not limited to, collagen, ADP, epinephrine, Ristocetin, Arachidonic Acid, thrombin and TRAP) (e.g. more light passes through a sample where there has been platelet aggregation as compared to a sample with no platelet aggregation) compared to a blank which is the donor's plasma with no platelets present (PPP or Platelet Poor Plasma). An agonist is a material that when added to platelet rich plasma, causes the platelets to aggregate. In LTA, the PRP is usually stirred in a cuvette at 37° C., and the cuvette sits between a light source and a photocell. After an agonist is added to platelet rich plasma (PRP), the platelets aggregate and absorb less light, so the light transmission increases and is detected by the photocell.

Light transmission aggregometry assays (LTAAs) generate data in the form of aggregation patterns. LTAAs generates parameters plotted on an x/y grid. The x axis is usually a linear time base (typically—minutes). The y axis is a logarithmic scale based upon light transmittance. This light transmittance is equated to percent (%) aggregation.

As the LTAA pattern or curve is generated, various derived measurements are calculated, including Slope (Sa); Maximum Aggregation; Final Aggregation; Area Under the Curve (AUC), Area Under the Slope (AUS) and others.

Slope of Aggregation (Sa) is a measurement of the rate at which the reaction is proceeding.

Dilution Profile (DUP) is an incremental change in concentration of the reactants in a test mixture. In collagen testing, the DUP is comprised of the changes to the concentration of the collagen reagent used. Other dilution profiles may be defined and used in the analysis.

Slope of the Dilution Profile (Sd) is generally the regression analysis of the change in concentration.

Slope of the Reaction Profile (Sr) is generally the regression analysis of the change of reaction to change of dilution.

The regression analysis may be linear, polynomial or follow other models.

Area Under the Curve (AUC) is a receiver operating curve that is the calculated graphical volume from the start of the reaction to the end of the reaction as defined by the aggregation and slope of aggregation (Sa). This may be considered as the “Power” generated by the reaction.

Previously it was thought that there was an all or nothing response to aspirin—that is, either a patient was “normal” (i.e. average aspirin sensitive) in that a very significant amount of platelets were inhibited from aggregation after taking aspirin, or the patient was aspirin resistant (aspirin non-responsive) in that the aspirin had little to no effect on platelet aggregation inhibition even after taking increased doses of aspirin (all or virtually all of the patient's platelets still aggregated). However, using methods of the present invention, and specifically using synthetic collagen as opposed to biological source collagen, it has been determined that in reality, there is a range of aggregation that occurs across patients and what was thought as a bright line between normal aspirin sensitivity and aspirin non-responsiveness is really a sliding scale. Because the present invention utilizes synthetic collagen, which it turns out is much more sensitive, predictive and precise than biological collagen, and thus allows extremely low amounts of synthetic collagen to be used, the inventors were able to see that in reality, there was not an all or nothing response across patient populations. Instead, there are ranges of aggregation/platelet aggregation and these ranges can be used to characterize an individual as aspirin hypersensitive, average aspirin sensitive, and aspirin non-responsive or identify the individual as non-compliant in their aspirin therapy.

Using methods of the present invention, the inventors were able to discover that patients who had previously been characterized as being average aspirin sensitive, were actually slightly aspirin non-responsive. Further, the inventors were able to determine that some patients respond to aspirin so strongly that they have an almost complete inhibition of platelet aggregation when taking aspirin, which may lead to bleeding problems that have been exhibited in a significant population of patients on aspirin therapy. Further, the inventors were able to observe that individuals, who were thought to be aspirin non-responsive, actually do have some platelet aggregation inhibition after taking aspirin, although not nearly at the levels of the average/normal aspirin sensitive individual. Using previous aggregation testing methods with biological collagen only provided a yes-no response (i.e. platelets aggregated or they did not aggregate). However, using the current inventive methods that involve the use of synthetic collagen (at much lower concentrations of biological collagen), the inventors surprisingly discovered that previous tests using biological collagen in some cases caused the wrong diagnosis (which could result in a major adverse cardiovascular event) and at the very least failed to provide correct or accurate information about the degree of aspirin sensitivity and compliance. As such, the present inventors believe that the use of biological collagen can now be understood to be the reason that various studies and clinical trials of aspirin efficacy have had inconsistent results.

Further, the present invention provides embodiments that capitalize on the sensitivity of the synthetic collagen and thus, employs the use of very low doses across multiple dilutions of synthetic collagen (referred to herein as “dilution profiles”) to aid a physician in determining not only whether a donor is aspirin sensitive or not, but to further understand a donor's aspirin sensitivity status (e.g. the degree to which a donor is aspirin sensitive or non-responsive) as well as compliance with the aspirin therapy.

This information may be useful for the physician to determine an appropriate dose of aspirin and the effective dosing schedule for the prescribed therapeutic regimen to be effective, or perhaps whether a second or third therapeutic medicine is required, or whether to consider abandoning the use of aspirin altogether for an alternative therapy. For instance, if an individual turns out to be aspirin hypersensitive or on the high end of average aspirin sensitivity, the physician may lower the dose of the aspirin than the average “low dose aspirin” therapy regimen. Low-dose aspirin (81 mg) is the most common dose used to prevent a heart attack or a stroke. However, the dose for daily aspirin can range from 81 mg to 325 mg. Tablets marketed as “low-dose aspirin” contain 81 mg aspirin. One adult-strength table contains about 325 mg aspirin. The physician could then continue to monitor the patient's aspirin sensitivity status during the aspirin therapy to determine if additional modifications to the aspirin dose are necessary, as well as monitor patient compliance. Aspirin pharmacodynamics confirm that the low dose −81 mg—provides complete and effective protection as long as the patient adheres to the dosing schedule. This also reduces the risk of side effects including gastritis and bleeding episodes that are more frequent when higher doses are administered. The physician could re-check the patient's aspirin sensitivity status using methods of the present invention to determine if the new doses were having the desired effect and could then either tweak the aspirin dose accordingly or perhaps even discontinue aspirin and try another anti-platelet medication if the aspirin was not achieving the desired result.

There have been reports in the literature that indicate a patient's response to aspirin can, under certain circumstances, change or be changed by medications prescribed for other conditions or even self-administered over the counter drugs such as ibuprofen. The present invention is suitable for determining a change in aspirin responsiveness. However, the status of an aspirin insensitive (non-responder) individual cannot change by increasing the dose of the aspirin. Accordingly, in these situations, an alternative anti-platelet therapy may be chosen.

Further capitalizing on the sensitivity of synthetic collagen and further using the dilution profile concept, the present invention also provides embodiments where a donor's aspirin sensitivity can be predicted even before the donor ingests aspirin. The present invention allowed the investigators to discover that aspirin non-responder (resistant) donors had a distinct response to LTAAs run over varying low concentrations of synthetic collagen, which could be used to diagnose a donor's response to aspirin. This and other embodiments are discussed more fully herein below.

As mentioned above, LTAAs use Platelet Rich Plasma (PRP), which is prepared from anti-coagulated whole blood. The donor's blood is collected and spun down to obtain the PRP. Since platelets are very sensitive and can be readily activated during the preparation of PRP, the donor's blood is usually collected in a tube containing a particular anticoagulant. For example, venous blood is obtained and collected into 3.2%/(0.109M) sodium citrate in a ratio of 1:9 (1 part anticoagulant to 9 parts blood). Whole blood samples should be processed within 4 hours of collection and blood samples for platelet aggregation testing must be stored at room temperature as cooling the platelets can lead to activation and erroneous test results. PRP is usually prepared by centrifugation at 20° C. for 10-15 minutes at 150-200 g. The PRP is carefully removed and placed into a stoppered plastic tube. PRP must be stored at room temperature.

Platelet Poor Plasma (PPP) can be then prepared by further centrifugation of the remaining plasma at 2700 g for 15 minutes. Platelet Poor Plasma (PPP) contains no platelets or other cellular material, and is often used as a blank in LTA sample analyses. In certain embodiments a special centrifuge that can generate PRP and PPP in about 5 minutes instead of the typical 45-60 minutes is employed. This makes the LTAA even more practical for emergency situations or for a high throughput clinical setting.

Addition of a platelet agonist to the PRP usually leads to platelet activation, and leads to a change in their shape from discoid to spiny spheres, which is associated with a transient increase in optical density. Exceptions to this are epinephrine in which there is no shape change, and ristocetin, which causes platelet agglutination rather than aggregation, i.e. there is no binding of fibrinogen.

Agonists are usually classified as strong agonists or weak agonists. Strong Agonists (e.g. Collagen, thrombin, TRAP, high concentration ADP, and U46619 (an analog of TxA2)) directly induce platelet aggregation, TxA2 synthesis and platelet granule secretion. Weak Agonists (e.g. low concentration ADP & epinephrine) induce platelet aggregation without inducing secretion.

In general, LTAAs are performed at 37° C. The aggregometer is calibrated by: 1) a cuvette containing PRP, which equates to 0% light transmission; and 2) a second cuvette containing PPP, which equates to 100% light transmission. Since platelets will normally only aggregate if they are activated (with an agonist) and are in contact with each other, they must be stirred whilst testing is taking place. Absence of stirring will lead to an absence of, or at least a significant reduction in, aggregation, resulting in erroneous test results which may then cause inappropriate care or treatment.

In certain embodiments, Bio/Data's PAP 8E LTA is employed (See U.S. Pat. No. 7,453,555) as the LTA used in the LTAAs of the present invention.

Normally a test for spontaneous platelet aggregation (“SPA”) is performed as part of a testing regimen. SPA is rare in healthy individuals, but occurs in people with hyperactive platelets, and is also recognized as a marker for some pro-thrombotic conditions, in some cases of von Willebrand Disease (vWD), in some patients with diabetes, in some lipid disorders and in a variety of other disorders. The presence of SPA is tested by placing undiluted PRP in the aggregometer and stirring for 15 minutes. In cases of SPA, dilution of the PRP may abolish this and if the platelet count remains >200×10⁹/L then aggregation testing can proceed.

In general about 225 μL of PRP is added to the aggregometry cuvette and warmed at 37° C. Then 25 μL of the agonist is added and the response recorded. The typical readouts or responses recorded include primary aggregation (“PA”) (which usually provides a value indicating the amount of aggregation), primary slope (“PS”)(which usually provides a value relating to the speed of aggregation) and area under the curve (“AUC”)(which generally provides a value relating to a combination of PA and PS). Aggregometry instruments used in the field typically will provide these readouts along with a pictorial graph of the aggregation. Each aggregometer or system calculates the values a bit differently and may use a proprietary formulae embedded in the system software.

For example, % maximal aggregation may be calculated by measuring the distance between the baseline [0% aggregation−platelet rich plasma] and platelet poor plasma [100% aggregation] [Y] and dividing this number by the maximal aggregation [X]. So if the Y=100 mm and X=89 mm then percentage maximal aggregation=X/Y=89%.

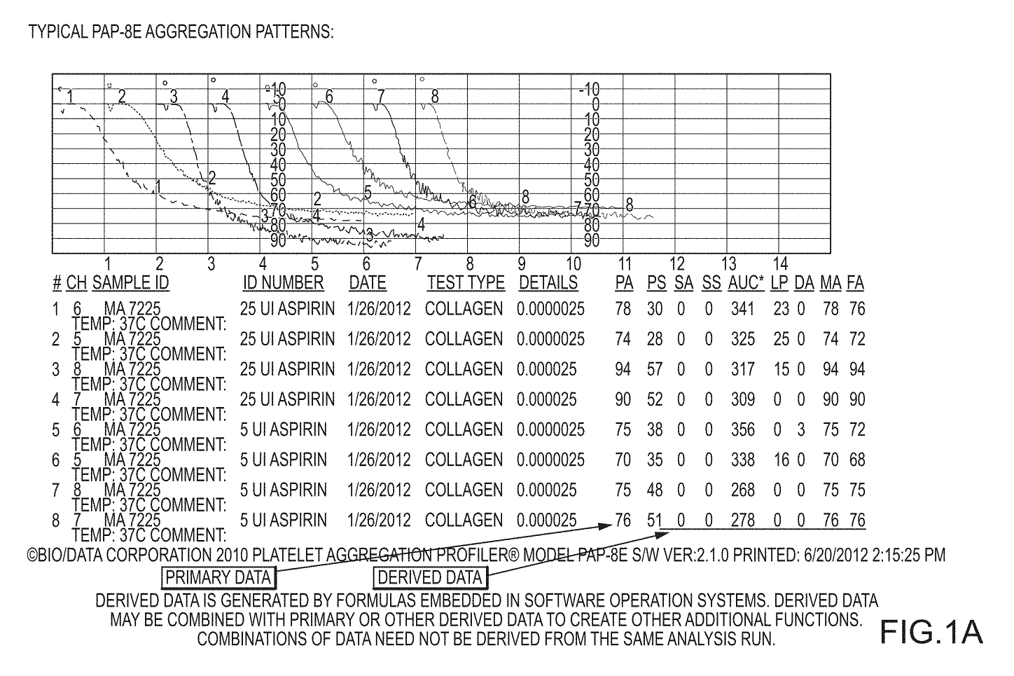

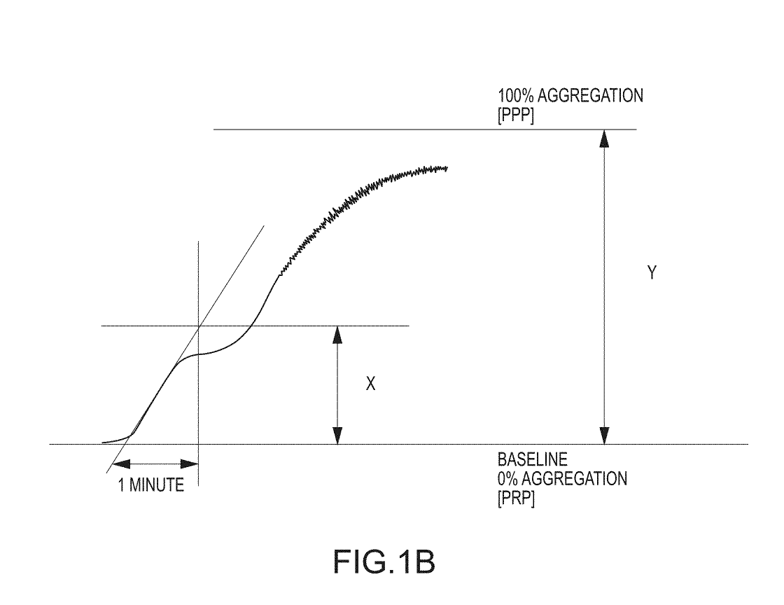

One method of calculating slope is described below and refers to FIG. 1b. To calculate the slope: 1) Draw a line at a tangent to the aggregation curve. 2) Determine how many millimeters [mm] the chart recorder records in 1 minute. 3) Measure in mm from the point where the tangent intersects the baseline to the distance equal to 1 minute. 4) Draw a line perpendicular to the baseline from the ‘1 minute’ point to the intersect point of the tangent. 5) Measure the distance [in mm] covered from the baseline to the intersect point [X]. 6) Derive the maximal height of the aggregation [100% aggregation or maximal aggregation] from the y-axis [Y]. Divide X/Y to calculate the slope or rate of aggregation. In the example above, if X=23 mm and Y=97 mm, the slope is X/Y=0.24

The present invention utilizes synthetic collagen as the agonist instead of collagen obtained from biological sources in the assays including flow cytometry and light transmission aggregometry assays (“LTAA”). The use of synthetic collagen provides unexpected benefits over the use of biological collagen, which is described herein.

One method of the present invention involves performing one or more aggregation assays, such as_light transmission assays whereby a first platelet rich sample is obtained from a donor and is combined with synthetic collagen to form a first treated sample. For this first treated sample, the donor has not ingested aspirin for a time period of about 24 hours, preferably 72-96 hours, and in some cases, preferably 168 hours. The idea is to make sure that the donor will not have any aspirin in his system to affect the platelet aggregation tests. The sample is then tested for platelet aggregation using such devices as an LTA aggregometer or a flow cytometry device) to obtain-a first readout to determine the donor's baseline level in the absence of ingested aspirin. In certain embodiments, an initial assay may be performed to check for spontaneous aggregation (SPA). Saline is added instead of the synthetic collagen to see if there is any aggregation. This tests for whether the platelets have any inherent hyperactivity.

Then the donor is given aspirin and a time period sufficient to allow the aspirin to be metabolized (e.g. at least about 2 hours to about 16 hours) is allowed to pass before a second platelet rich plasma sample is obtained from the donor. With small doses (about less than 250 mg, all pathways proceed by first-order kinetics, which an elimination of half-life about 4 hours. When higher doses of salicylate are ingested (e.g. more than 4.0 g), the half-life becomes much longer (15-30 hours). This same lengthening of the half life occurs in the elderly, and in patients with compromised kidney function, etc. A second assay is performed on the second platelet rich plasma sample by treating it with synthetic collagen to form a second treated sample. Aggregation of the second treated sample is measured to obtain a second readout. In certain embodiments, instead of having the donor ingest aspirin, the plasma is treated with aspirin.

The baseline level readout in the absence of ingested aspirin is compared with the second treated sample readout (obtained after aspirin ingestion/or having the sample aspirinated) and the results of this comparison will determine the donor's platelet aspirin response status. For example, if the donor shows a significant reduction in platelet aggregation after aspirin ingestion (in the second sample) as compared to the baseline sample, then the donor may be characterized as normal or average aspirin sensitive. If the donor shows very little difference in the platelet aggregation after taking aspirin (i.e. the platelets still aggregated after the donor ingested aspirin), then the donor may be characterized as aspirin non-responsive. If the donor showed an almost complete lack of platelet aggregation after ingesting aspirin, then the donor may be characterized as aspirin hypersensitive.

In the methods/assays of the present invention, instead of having the patient ingest aspirin and thereafter take a blood sample, a sample of blood can be taken before any aspirin is ingested and the sample can be “aspirinated” (or “aspirinized”)(that is, an aspirin solution (may also be the lysine salt of aspirin or Aspisol®) is added to the PRP and then tested). It has been determined that in the all of the methods/assays of the present, either the patient can ingest the aspirin or the sample can be aspirinated. This can speed up the testing because the patient does not need to ingest the aspirin and have time pass to allow the aspirin to get into the patient's system. Instead, the blood is drawn and a PRP sample is obtained, and one part is aspirinated and the other part is not, thus also allowing the two samples to be tested side by side.

When the aggregation assay is LTAA, the readout may be slope, primary aggregation, area under the cover, or a combination thereof.

For example, when using the Bio/Data's PAP 8E aggregometer, and when using an “in-test” concentration of 30 ng/mL of synthetic collagen, for an aspirin hypersensitive donor, the baseline for PA will range from 60% to 95%. The baseline for PS will range from 30 to 70. The baseline for AUC will range from 300 to 600. After aspirin, the AUC will range from 200 to 450. PS and PA will be different from their respective baselines. Aspirin sensitive and aspirin non-responders will show differences from baselines; sensitive donors will show less aggregation. After aspirin the PS will range from 25 to 60.

Varying amounts of synthetic collagen can be used in the assays. The amount of synthetic collagen used is about 1,000 fold less than what is generally used when performing LTAAs with biological source collagen. For example, usually LTAAs using calf skin biological collagen generally use 0.19 mg/mL (milligrams/mL) collagen (as the “in-test” concentration); and LTAAs using equine tendon collagen generally use 2.0 μg/mL (micrograms/mL) collagen in the LTAA test (as the “in-test” concentration), whereas generally the methods of the present invention utilize from about 0.05 ng/mL to about 50 ng/mL (nanogram/mL) of synthetic collagen in each LTAA test (as the “in-test” concentration). In certain embodiments the amount of synthetic collagen used will range from about 500.0 ng down to about 0.50 ng/mL present in each LTAA test (i.e. in each cuvette) (as the “in-test” concentration). In other embodiments, the present invention utilizes from about 500.0 ng/mL to about 5.00 ng/mL in each LTAA (as the “in-test” concentration). In other embodiments, the present invention utilizes from about 50.0 ng/mL to about 0.50 ng/mL in each LTAA (as the “in-test” concentration). In other embodiments, the amount of synthetic collagen for each LTAA test rill range from about 0.05 ng/mL to about 50 ng/mL (as the “in-test” concentration).

When testing platelet aggregation using flow cytometry, the usual concentrations of biological collagen range from 0.01-100 μg/mL, with 20 μg/mL seems to be most common. However, using synthetic collagen, the amounts used are much lower, ranging from about 2.0 ng/mL to about 640 ng/mL.

In certain embodiments, when the aggregation assay uses, the amount of synthetic collagen used as in the in-test collagen ranges from 2 ng/mL to 64 ng/mL. In certain embodiments, the amounts of in-test synthetic collagen ranges from 4 ng/mL to 64 ng/mL; from 6 to 64 ng/mL; from 8 ng/mL to 64 ng/mL; from 2 ng/mL to 100 ng/mL; from 4 ng/mL to 100 ng/mL; from 6 to 100 ng/mL; from 8 to 100 ng/mL; and any subset of ranges or individual numbers from 2 ng/mL to 100 ng/mL. In certain embodiments the amounts of in test synthetic collagen is 2 ng/mL, 4 ng/mL, 6 ng/mL, 8 ng/mL, 16 ng/mL, 32, ng/mL and/or 64 ng/mL. In certain embodiments the amount of in-test synthetic collagen is any number in the range of 2 ng/mL to 100 ng/mL, such as, but not limited to 2 ng/mL, 3, 4, 5, 6, 7, 8 . . . 95, 96, 97, 98, 99, or 100 ng/mL.

Although there are ranges included hereinabove, the present invention is not limited by the recitation of the first and last endpoint to only mean the first and last, but expressly includes the first and last endpoint as well as all of the concentrations within the endpoints. It would be just too cumbersome herein to list every concentration about that falls within the recited ranges. The inventors have contemplated using more than one concentration, and more than one range as well as more than one concentration within the recited range. In some cases the assays have used as many as eight (8) different concentrations within a recited range. For example, as discussed in more detail herein below, LTAAs have been run with many different dilutions and tested these dilutions in profiles. For example but not limited to, the dilution profiles have been run with the following different “in-test” dilutions: 500 ng/mL; 50.0 ng/mL; 25.0 ng/mL; 10.0 ng/mL; 5.0 ng/mL; 2.50 ng/mL; 1.0 ng/mL; 0.50 ng/mL, and 0.25 ng/mL.

The ability to use such low concentrations of collagen is only available with the synthetic collagen. When the scientists tried to dilute biological collagen down to similar low concentrations, it became physically impossible to dilute down the biological collagen to the levels anywhere close to that used in the LTAAs of the present invention with synthetic collagen. Biological collagen is an insoluble, viscous, heterogeneous material, and has long structured fibrous proteins wound into a triple helix. These physical properties precluded the ability to dilute the biological collagen to any low concentration even 100 fold close to the synthetic collagen. Further the LTAAs did not work (no aggregation occurred) when using calf skin collagen at a concentration when using equine tendon collagen a little lower than 2.0 μg/mL (micrograms/mL).

By using these extremely low amounts of synthetic collagen, a more sensitive assay is obtained than what can be achieved using biological collagen. For example, one particular donor was previously thought to have normal/average aspirin sensitivity (that is, aspirin would inhibit platelet aggregation) based on the results of LTAAs performed using biological collagen. However, when this donor's platelets were tested using methods of the present invention, it was discovered in actuality that the donor was aspirin non-responsive. These tests are discussed in more detail below.

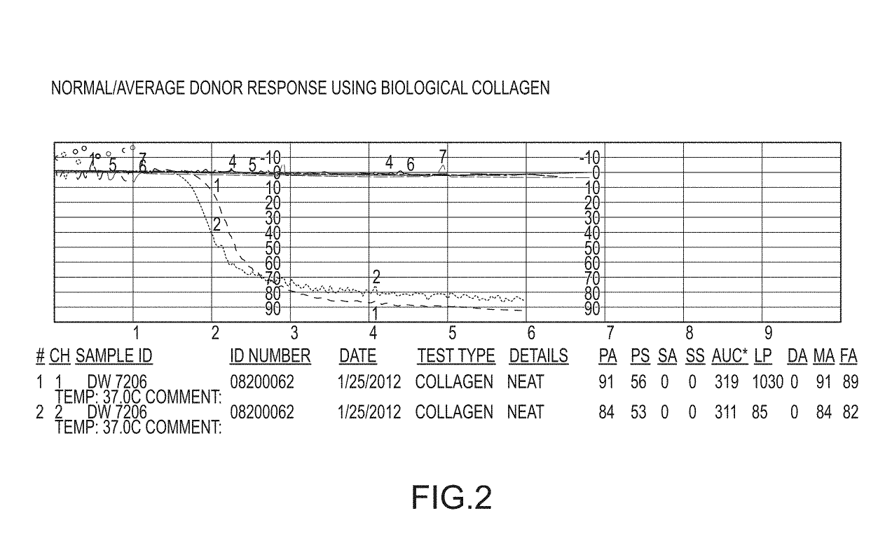

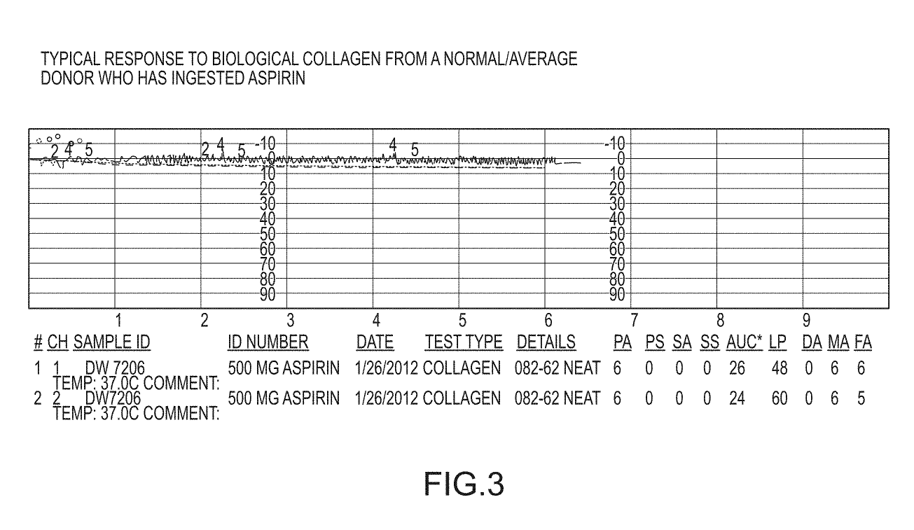

FIG. 2 shows the results of a test using platelets obtained from normal/average aspirin sensitive (donor 7206) who has a normal/average aspirin response and using a biological collagen (0.019 mg/mL BDC (calf skin collagen) and 0.2 μg/mL Chrono-Log collagen). Two separate samples were run and they each show a high percentage of platelet aggregation. Primary aggregation (“PA”) was 91 and 84; Primary slope (“PS”) (which is the rate of aggregation) was 56 and 53. Area under the curve (“AUC”) was 319 and 311. Testing the same donor's platelets with synthetic collagen at concentrations 25.0 ng/mL to 2.5 ng/mL also showed a high percentage of platelet aggregation. (Results not shown). The same donor (donor 7206) was then given 500 mg of aspirin and later the platelets were obtained and tested in a LTAA with 0.019 mg/mL, 0.085 mg/mL, and 0.2 μg/mL of biological collagen. As would be expected, there is a significant reduction in platelet aggregation shown. See FIG. 3. Using the PAP 8E aggregometer, the PA was 6 and the PS was 0 and the AUC was 26 and 24.

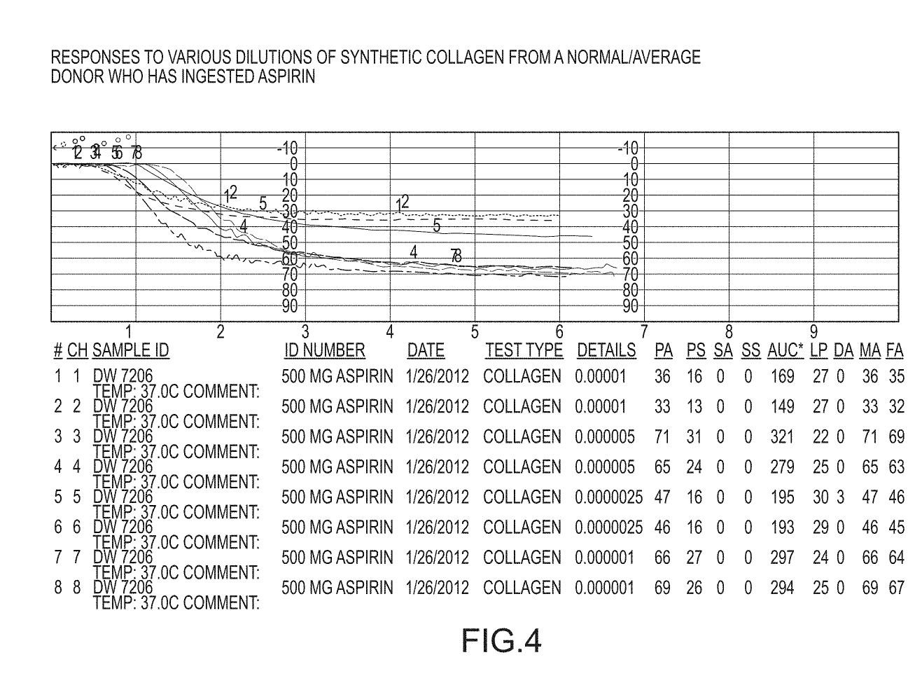

Next, synthetic collagen was tested with the normal/average donor's platelets (donor 7206) after the donor ingested 500 mg of aspirin. In this test, various amounts of synthetic collagen were tested (ranging from 0.00001 mg to 0.000001 mg)(i.e. from 10.0 ng to 1.0 ng). Even using these very low amounts of synthetic collagen, one can see that some levels of platelet aggregation were visible even after the donor ingested aspirin. See FIG. 4. With synthetic collagen and using far much less than the amount of biological collagen used, platelet aggregation can be seen. The amount of platelet aggregation that occurs after aspirin ingestion in the normal/average donor is less than what was seen before aspirin ingestion (as expected). FIG. 4 shows that varying amounts of aggregation observed depended upon the concentration of synthetic collagen used. One can see the near stoichiometric relationship between synthetic collagen dilutions and platelet aggregation response.

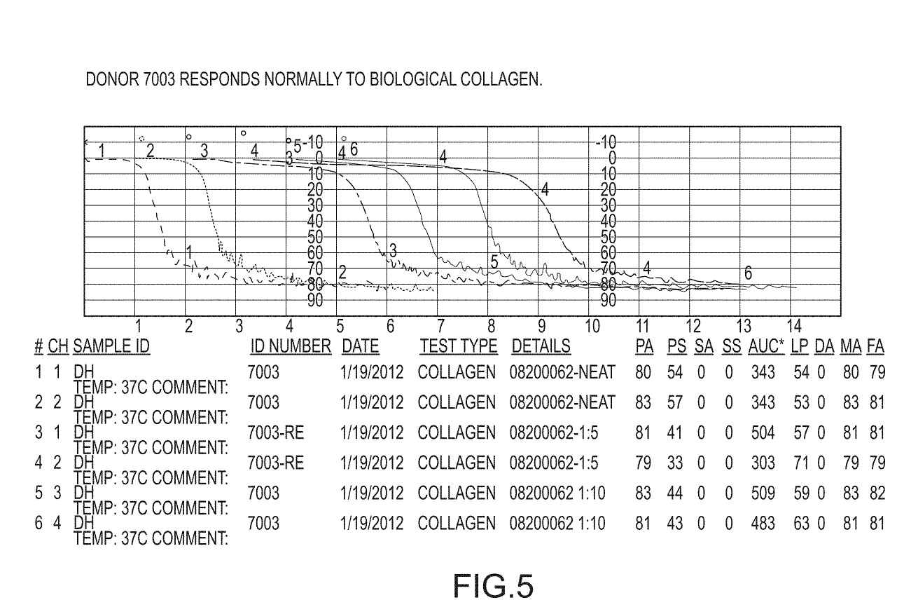

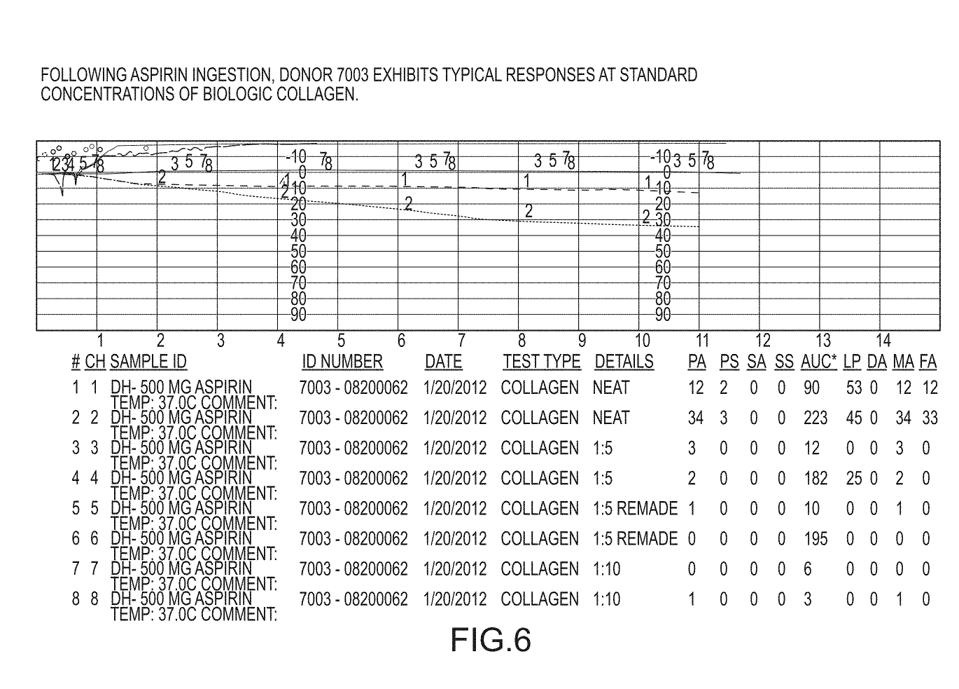

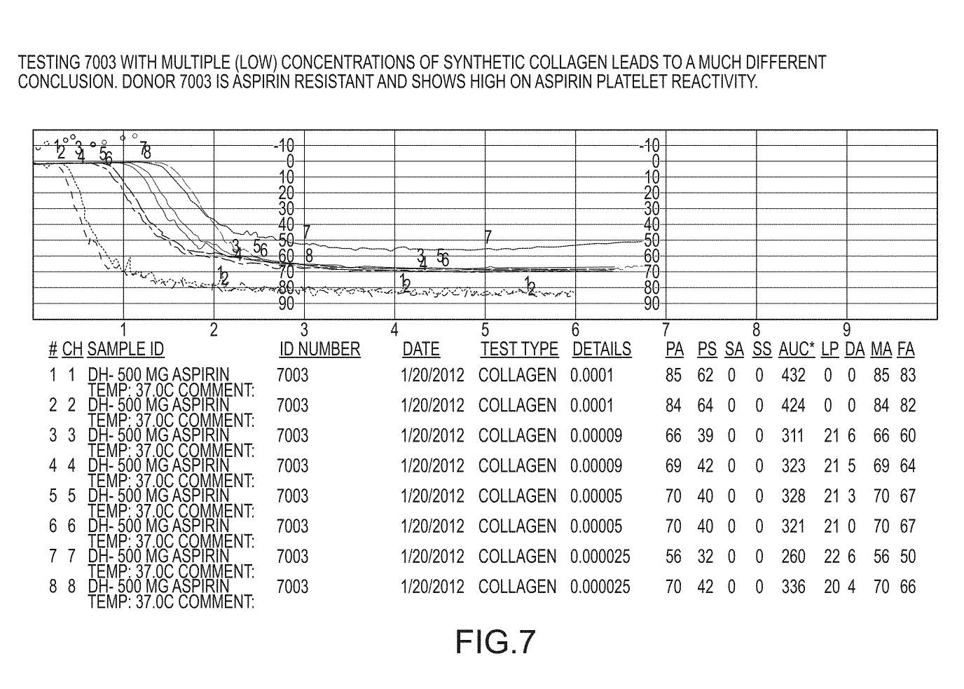

Next, another donor was tested (donor 7003). It was previously believed (based on LTA tests run with biological collagen) that this donor had a normal/average aspirin sensitivity (after ingestion of aspirin, a normal amount of platelet aggregation was measured using biological collagen). FIG. 5 shows the results of a LTAAs run on donor 7003 platelets before ingestion of aspirin using 0.019 mg/mL, 0.085 mg/mL, and 0.2 μg/mL biological collagen. As was expected, platelet aggregation was seen. Similarly, LTAAs run on donor 7003 platelets before ingestion of aspirin using 0.25 to 25 ng/mL synthetic collagen also shows platelet aggregation. When donor 7003 is given aspirin and the platelets are again tested in LTAAs with biological collagen, there is a huge reduction in aggregation (little to no aggregation was observed), thus causing the diagnostician to believe that donor 7003 has a normal/average aspirin sensitivity. See FIG. 6. However, when LTAAs were run on donor 7003 platelets after aspirin ingestion, using various low amounts (i.e. from 25.0 ng/mL to 2.50 ng/mL) of synthetic collagen, aggregation is observed. See FIG. 7. Thus, donor 7003 does not have a normal/average aspirin sensitivity, but rather exhibits some degree of aspirin non-responsiveness. FIG. 7 shows that donor 7003 has “high on aspirin platelet reactivity” (which means that even with aspirin, the donor's platelets are still sticky and aggregate to some extent). Thus, this test shows that synthetic collagen is much more sensitive and specific in detecting aspirin non-responsiveness than biological collagen, even using much lower concentrations of synthetic collagen than what was used in the biological collagen assays.

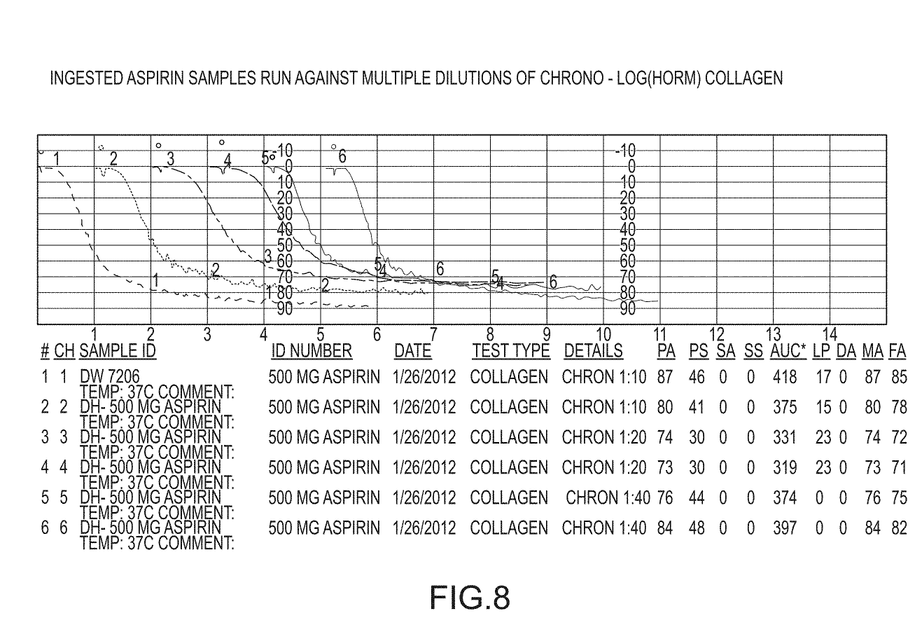

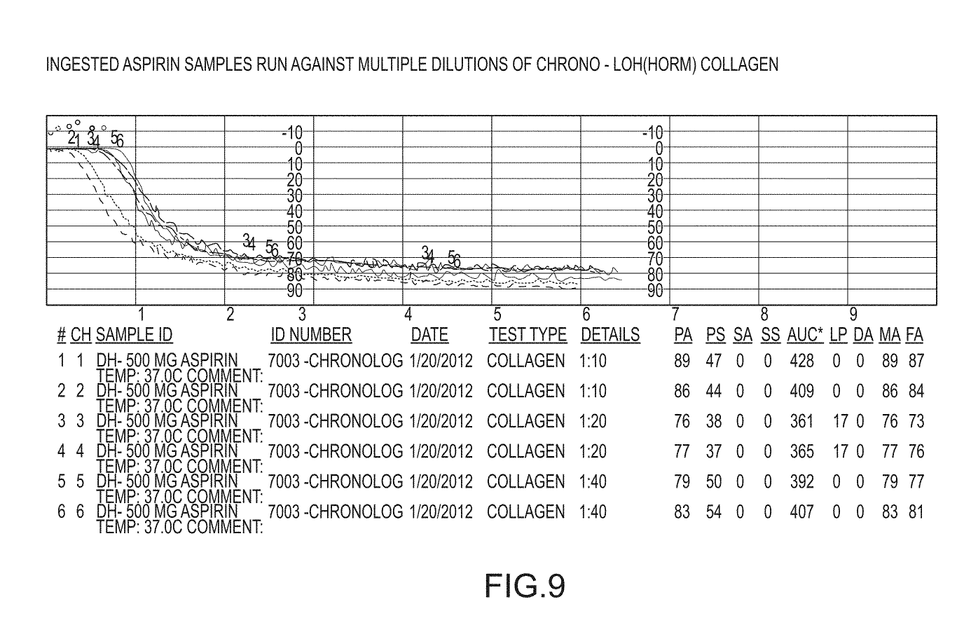

In other LTAA tests using biological collagen at multiple concentrations, the results obtained showed that the biological collagen could not distinguish between the normal/average sensitivity donor's platelets (7206) and the aspirin non-responsive donor's platelets (7003). FIG. 8 shows LTAAs run against normal/average aspirin sensitive donor 7206 with multiple dilutions of biological collagen after the donor ingested aspirin. In all dilutions, platelet aggregation is seen. FIG. 9 shows LTAAs run against aspirin non-responsive donor 7003 with multiple dilutions of biological collagen after the donor ingested aspirin. In all dilutions, platelet aggregation is seen. Thus, these figures show that LTAAs using low amounts of biological collagen do not work in that they cannot distinguish between a normal/average aspirin sensitive platelet donor and an aspirin non-responsive platelet donor.

In another embodiment of the invention, more than two reactions (more than just the baseline (before aspirin ingestion) and the after aspirin test) are run. A series of assays are run using multiple differing low amounts of synthetic collagen. This is referred to herein as the dilution profile assays, dilution profile LTAA, or dilution profiles. In this embodiment, multiple different PRP samples are obtained from the donor before aspirin ingestion (to obtain a baseline dilution profile) and after aspirin ingestion or after aspirinating the sample (to obtain a post aspirin dilution profile). Each pre-aspirin donor platelet sample is mixed with a different amount of synthetic collagen and an aggregation assay (such as an LTAA) is performed on each sample to obtain a baseline dilution profile over the range of concentrations. Then the donor is given aspirin and sufficient time is allowed to pass to ensure the aspirin has been metabolized (or the sample is aspirinated). Multiple PRP samples are obtained from the donor and mixed with different amounts of synthetic collagen. Aggregation assays (e.g. LTAAs or flow cytometry) are performed on each sample to obtain a post-aspirin dilution profile. The same concentrations of synthetic collagen that were used in the pre-aspirin baseline aggregation assays are preferably used in the post-aspirin aggregation assays. The results are analyzed (such as the change in PA, PS or AUC or a combination thereof between the pre- and post-aspirin LTAAs, as well as changes in the PA, PS or AUC or a combination thereof over the differing amounts of synthetic collagen) and studied to determine the donor's aspirin sensitivity response (whether aspirin hypersensitive, aspirin sensitive or aspirin non-responsive and the degree of sensitivity therein or non compliance). In certain embodiments, when the aggregation assay is LTAA, the results are analyzed using the aggregometer's proprietary algorithm embedded in system software, which makes the analysis and subsequent report easier for the diagnostician to interpret.

In other embodiments, the pre-aspirin baseline is established with one aggregation assay performed using one concentration of synthetic collagen in the aggregation assay on a pre-aspirin donor platelet sample, whereas multiple different concentrations of synthetic collagen are used in multiple aggregation assays to create the post-aspirin dilution profile assays In this case, the results are analyzed (such as the change in PA, PS or AUC or a combination thereof from differing amounts of synthetic collagen when the assay is LTAA) and studied, as well as compared against the baseline (pre-aspirin) aggregation assay to determine the donor's aspirin sensitivity response (whether aspirin hypersensitive, aspirin sensitive or aspirin non-responsive and the degree of sensitivity therein).

In other embodiments, a pre-aspirin baseline or pre-aspirin dilution profile is not obtained. This may be useful in the emergency clinical setting when it is not feasible to obtain a pre-aspirin baseline or whether one cannot determine from the patient whether he or she has been on aspirin therapy. In this embodiment, multiple different platelet rich plasma samples are obtained from the donor and each are mixed independently with a different synthetic collagen concentration to obtain multiple different treated samples for the dilution profile aggregation assays. Aggregation assays are performed for each of these samples to obtain an aggregation assay dilution profile over the range of different concentrations. The data is obtained and measured. In the case of LTAAs, the PA, PS or AUC or combinations therefore are obtained and analyzed over the different ranges of synthetic collagen. In certain embodiments, the results are analyzed using the aggregometer's proprietary algorithm embedded in system software.

The inventors have determined that this embodiment as well as other dilution profile embodiments can be used to predict the donor's platelet aspirin response. For individuals having an average or “normal” aspirin sensitivity, when looking at the slope, percentage aggregation or the AUC, for example, over the dilution profile, the slope, percentage aggregation or the AUC show a corresponding decrease along with the decrease in the amount of synthetic collagen used. Thus, for example, within a given range of various dilutions of synthetic collagen, as the concentration goes down, so will the slope, percentage aggregation, and the AUC. There seems to be an almost linear decrease in slope, percentage aggregation and AUC that runs almost parallel or has almost a direct correlation with the concentration of synthetic collagen. On the other hand, for individuals who are aspirin non-responders, instead of having a slope, percentage aggregation, and AUC that has a linear-like decrease that corresponds with the decrease in synthetic collagen, there is a point along the dilution profile where there is an increase in slope, an unexpected temporary increase in percentage aggregation and/or a temporary increase in AUC when there should be a decrease (because the concentration of synthetic collagen decreases). Then, as the dilution profile continues to decrease, the slope and AUC “bounce back” down to where it should be (based on the dilution of synthetic collagen) and where it was before the temporary increase and then continues along decreasing.

Aspirin hypersensitive individuals will show increase in PA, PS and AUC compared to expected/normal results.

A review of certain figures discussed below, provide a further demonstration of the above embodiment, and also shows that what was predicted in LTAAs before aspirin ingestion using the dilution profile LTAAs, was confirmed later when the donor ingested aspirin and LTAAs were performed.

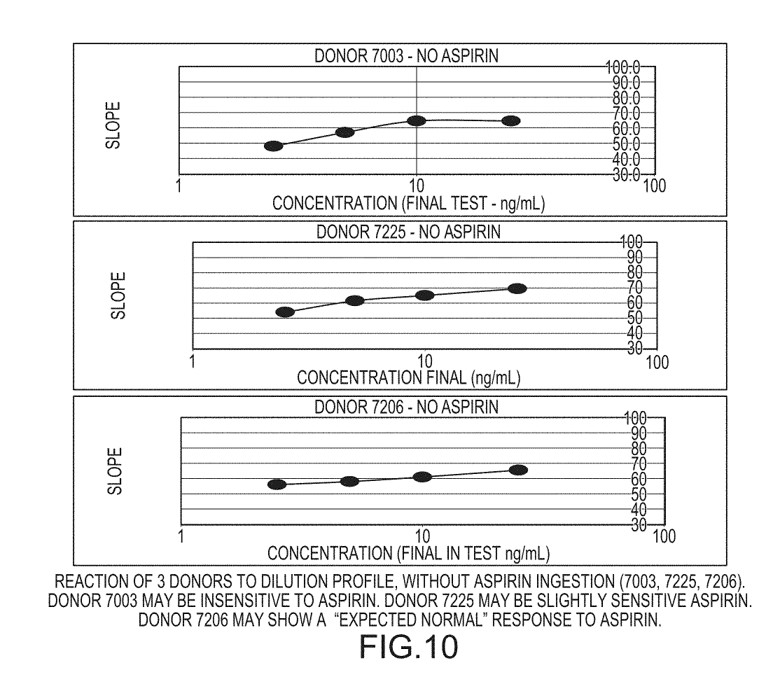

FIG. 10 shows slopes from dilution profile LTAAs run using three different donor platelets using synthetic collagen. Four different concentrations were used 2.5 ng/mL, 5.0 ng/mL 10.0 ng/mL and 25 ng/mL. One familiar with the responses to synthetic collagen would expect that a normal response (an individual with an average aspirin sensitivity) to have a linear dilution profile (such as seen with donor 7206)—that is, as the concentration goes down, the slope decreases. Donor 7003's response is definitely non-linear, thus indicating that donor 7003 does not have a normal response and thus will not have an average aspirin sensitivity.

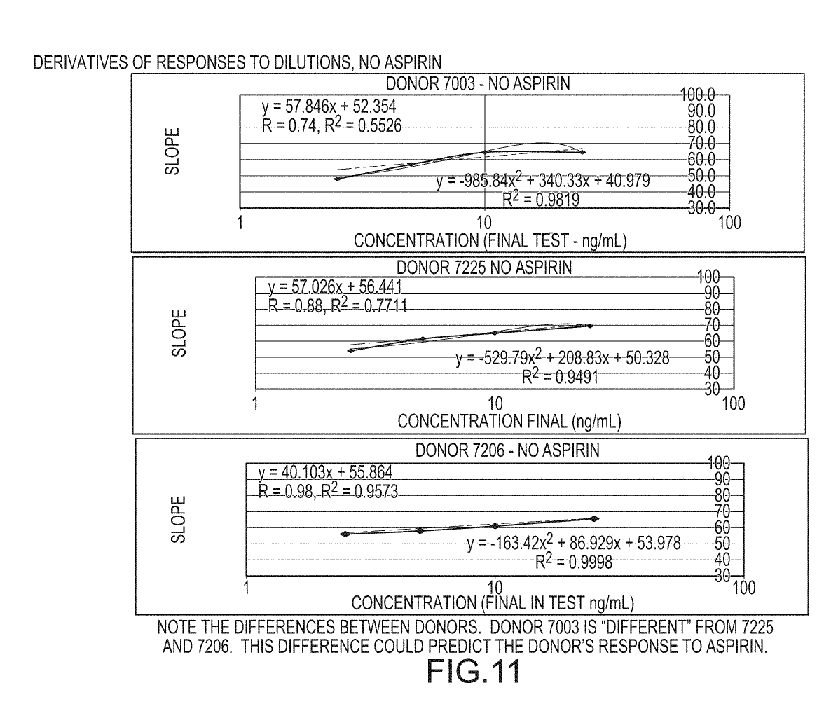

FIG. 11 provides the results of LTAAs using synthetic collagen. The aspirin response status of the three donors is as follows: donor 7003 may be insensitive to aspirin; donor 7225 may be slightly sensitive to aspirin and donor 7206 may show an expected normal response to aspirin. These statements are conclusions made from previous work with synthetic collagen using both pre and post aspirin LTAAs. However, what is interesting that even though the tests reported in this figure are performed on platelets with no aspirin, these slopes provide an insight into the donor's status even before an LTAA run post aspirin is conducted. This can only achieved with synthetic collagen and low concentration amounts. The shape of the slope on these LTAAs run over a dilution profile of synthetic collagen (multiple different LTAAs run on each donor's platelets using various different low concentration amounts of synthetic collagen) provide this key insight. It was expected that the slope would decrease as the concentration of synthetic collagen is lowered. This can be seen in donor 7206, which has been determined to be a normal response (would have an average/normal aspirin sensitivity). But the slope derived from the LTAAs run on donor 7003 platelets does not make a steady decrease corresponding with the dilution of the synthetic collagen. There is actually one point where the slope increases for a short period and then returns back down and then continues decreasing. This “hump” in the slope can be seen in FIG. 11 at a concentration somewhere between 100 ng/mL to 10 ng/mL (roughly 50 ng/mL). This phenomena is often referred to herein as the “bounce back.” The bounce back has also been seen when running other LTAAs with other dilution profile concentrations of synthetic collagen ranging from 50 ng/mL to 2.5 ng/mL, as well as ranging from 1 ng/mL to 100 ng/mL. The ability to make this prediction of a donor's response without having them ingest aspirin is a huge advantage of using synthetic collagen (and is not achievable using biological collagen). Further, this result was totally unexpected.

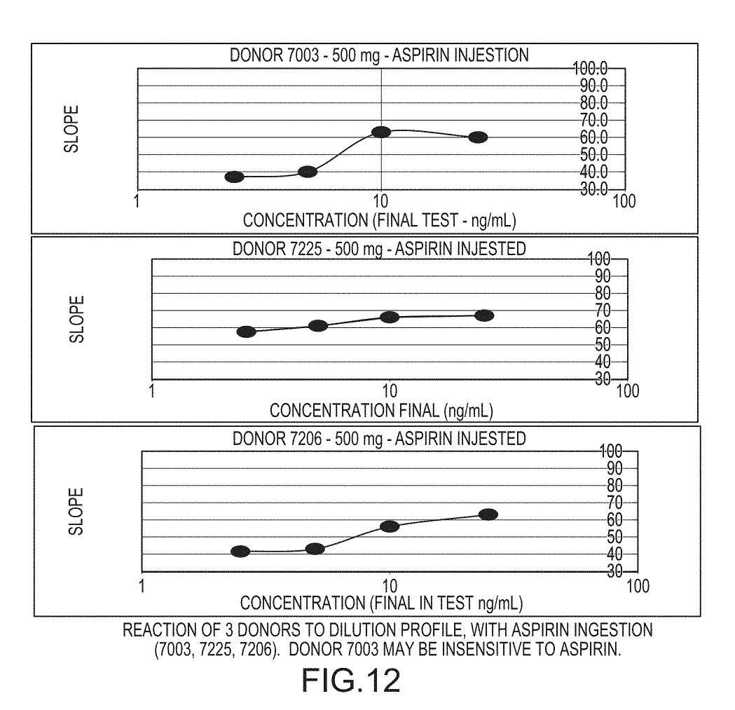

FIG. 12 provides the results of LTAAs using synthetic collagen. The fact that because Donor 7003 had this temporary rise and then fall in the slope even when the concentration of collagen was reduced from 100 ng/mL to 10 ng/mL allows one to make the prediction that donor 7003 is probably aspirin non-responsive. This is the “bounce back” phenomena. This figure shows the response slope after the donors ingested aspirin and it shows that it corresponds to the slope pattern when the donors had not ingested aspirin (as in FIG. 11). Thus, this figure provides confirmation the predicted donor's response as shown in FIG. 11 before aspirin was ingested.

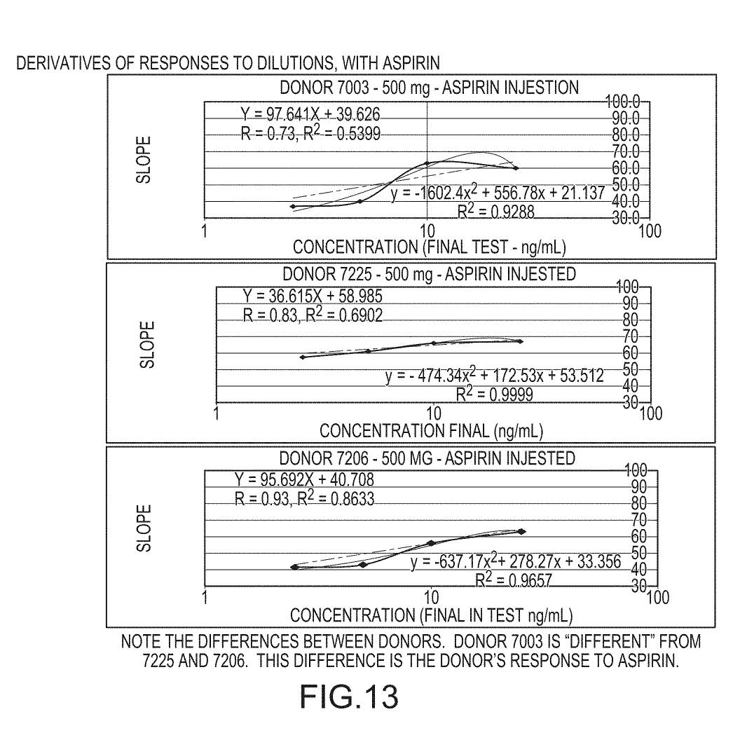

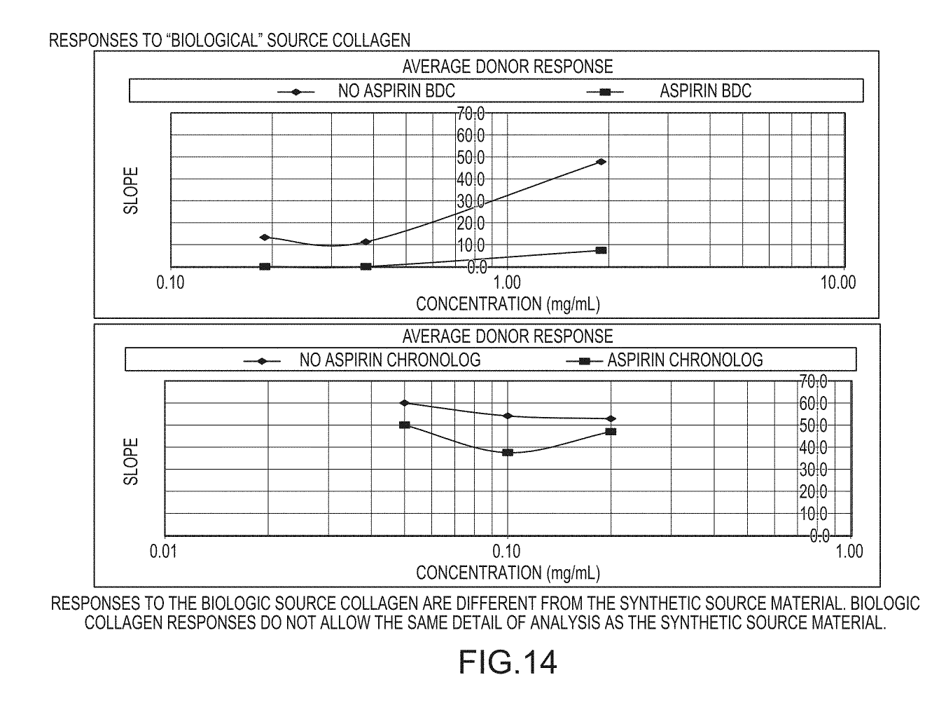

FIG. 13 provides the results of LTAAs using synthetic collagen. FIG. 13 shows the “bounce back” in donor 7003, as opposed to a more linear-like slope seen in donor 7225 and 7206.

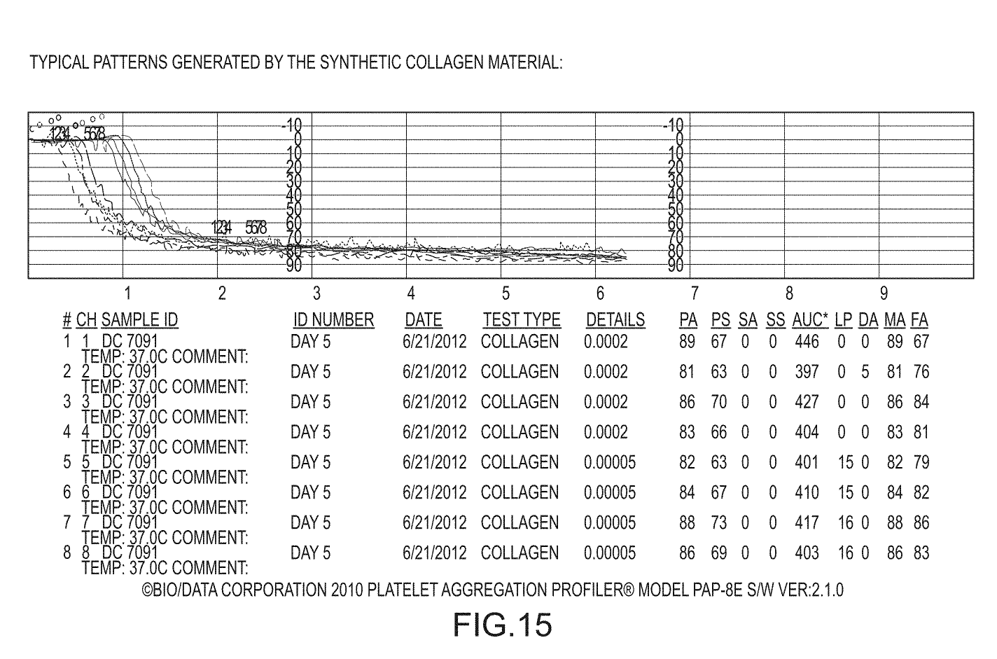

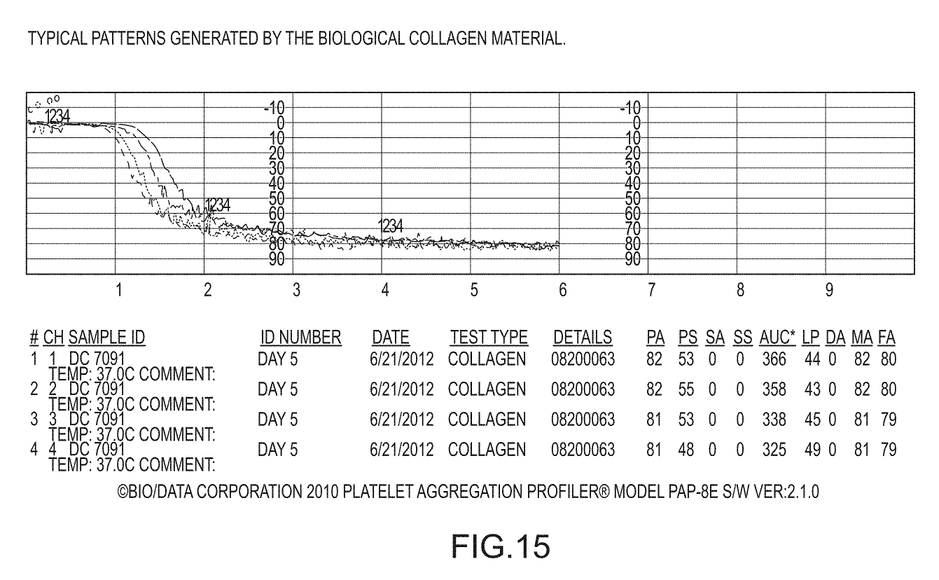

FIG. 15 shows that synthetic collagen at particular concentrations and biological collagen generate very similar looking curves, but the synthetic collagen is used at a fraction of the amount of biological collagen. In this figure, donor 7091 platelets were tested with 0.0002 mg/mL (i.e. 200 ng/mL) and 0.00005 mg/mL (i.e. 50 ng/mL)(final concentration) of synthetic collagen (i.e., the “in-test” concentration), whereas the same donor's platelets were tested with 0.19 mg/mL biological collagen. Results could not be obtained using similar low concentrations of biological collagen. Even though FIG. 15 shows that synthetic collagen and biological collagen generate very similar looking curves, the reported parameters for the synthetic collagen show clearly that: the AUC is significantly higher—suggesting much greater sensitivity.

In certain embodiments of the invention utilizing the dilution profile aggregation assay_concept, a series of 7, 6 or 5 different concentrations are used for the dilution profile, and in other embodiments, 4 different concentrations are used and yet in other embodiments, 3 or 2 different concentrations are used. Using too many different concentrations can make the test cumbersome and time consuming, whereas using too few concentrations reduces the amount of data obtained and limits the sensitivity analysis.

The range of synthetic collagen used is preferably within the “sensitive range,” which is defined herein as the range of concentrations in which in an average aspirin sensitive donor, the measured platelet activity/aggregation is reduced corresponding with decreasing amounts of synthetic collagen concentrations (e.g. the AUC and/or the slope decreases with the concentration of collagen). In certain embodiments the sensitive range is from about 2.0 ng/mL to about 640 ng/mL. In certain embodiments the sensitive range is from about 0.05 ng/mL to about 500 ng/mL. In certain embodiments the sensitive range is from about 2.0 ng/mL to about 250 ng/mL. In certain embodiments the sensitive range is from about 1.0 ng/mL to about 250 ng/mL. In certain embodiments the sensitive range is from about 2.0 ng/mL to about 250 ng/mL. In certain embodiments the sensitive range is from about 5.0 ng/mL to about 500 ng/mL. In certain embodiments the sensitive range is from about 5.0 ng/mL to about 250 ng/mL. In certain embodiments the sensitive range is from about 5.0 ng/mL to about 100 ng/mL. In certain embodiments the sensitive range is from about 2.0 ng/mL to about 100 ng/mL.

In certain embodiments, the different synthetic collagen dilution amounts comprise multiple different synthetic collagen amounts chosen from within the concentration range from about 0.050 ng/mL to about 50.0 ng/mL.

For example, in certain embodiment there are seven different synthetic collagen amounts as follows:
i) about 50.0 ng/mL;
ii) about 25.0 ng/mL;
iii) about 10.0 ng/mL;
iv) about 5.0 ng/mL;
v) about 2.50 ng/mL;
vi) about 1.0 ng/mL; and
vii) about 0.50 ng/mL.

In other embodiments there are five different synthetic collagen amounts chosen from within the concentration range from about 50.0 ng/mL to about 0.050 ng/mL. For example in certain embodiments there are five different synthetic collagen amounts as follows: (all in ng/mL as the final “in-test” concentration): about 50.0; about 25.0; about 10.0; about 5.0; and about 2.5. In other embodiments there are five different synthetic collagen amounts as follows (all in ng/mL as the final “in-test” concentration): about 25.0; about 10.0; about 5.0; about 2.50; and about 1.0.

In other embodiments there are five different “in-test” synthetic collagen amounts that include one amount from within each of the following ranges: about 50.0 to about 25.0 ng/mL; about 25.0 to about 10.0 ng/mL; about 10.0 to about 5.00 ng/mL; about 5.00 to about 2.50 ng/mL; and about 2.50 to about 1.00 ng/mL).

In other embodiments there are five different “in-test” synthetic collagen amounts that include one amount from within each of the following ranges: about 6.0 to about 4.0 ng/mL; about 2.7 to about 2.3 ng/mL; about 1.5 to about 0.90 ng/mL; about 0.60 to about 0.40 ng/mL; and about 0.30 to about 0.20 ng/mL.

In other embodiments there are three different “in-test” synthetic collagen amounts chosen from within the concentration range of about 50.0 to about 25 ng/mL for the highest concentration of synthetic collagen and about 2.50 to about 0.50 ng/mL for the lowest concentration.

In other embodiments, there are three different “in-test” synthetic collagen amounts chosen from within the concentration range of about 6.0 to about 4.0 ng/mL for the highest concentration and from about 0.3 to about 0.1 ng/mL for the lowest concentration.

In another embodiment of the invention, there is provided a method of testing/monitoring patient compliance in taking the prescribed doses of aspirin and residual platelet activity using assays discussed above with synthetic collagen. Non-compliance includes not taking the aspirin, not taking the proper dose or not staying with the effective dosing (time) schedule. It has been discovered that some biologically derived collagens are insensitive to aspirin so a test using these collagens as the agonist would not provide reliable test results. Further, recent studies have shown that a large problem in health care is patient noncompliance. Current thinking is that what was once thought to be aspirin resistance may instead be a manifestation of non-compliance complicated by the use of multiple, non-standardized laboratory tests to evaluate platelets inhibited response to aspirin.

Accordingly, to measure compliance the patient can be routinely tested, such as once a week, bi-monthly, monthly, every 3 months, etc, and the results compared against each other. If the aggregation results vary widely from one test to another, the patient can be further tested to determine if aspirin resistance has developed or the patient could be questioned as to his compliance in taking the prescribed doses of aspirin. If it is suspected that the patient has not been taking the aspirin, the patient's plasma can be treated with aspirin and then tested. If aggregation appears in the aspirinated sample, then it may be concluded that the patient had not been taking the aspirin as directed. In some cases, the patient may be taking the aspirin sporadically and not at the same time each day. The aggregation tests may reveal variability from test to test and this variability could be used as an indicator that the patient has not been following the prescribed regular dosing regimen (either not taking the dose every day or taking the dose at different times of the day). It has been found that a patient on aspirin therapy that does not comply with the therapy but not taking the aspirin every day or taking it at different times of the day actually puts the patient at a higher than baseline levels for risk of a thrombotic event. If aggregation does not appear in the aspirinated sample, it could be that the patient had developed aspirin resistance. Further testing could be performed to determine if the patient should be on a dual therapy of aspirin and a different anti-platelet medication or perhaps a regimen a different anti-platelet medication altogether without aspirin.

Synthetic Collagen

In certain embodiments the synthetic collagen is described in U.S. patent application Ser. No. 12/520,508, which is herein incorporated by reference in its entirety. In certain embodiments, the synthetic collagen is a synthetic collagen that has the ability to self-assemble into a triple helix to form fibrils, which allows the synthetic collagen to mimic type I collagen. In certain embodiments the synthetic collagen comprises a polypeptide having a peptide fragment represented by the formula (I)

-(Pro-X-Gly)ₙ (I)

wherein X represents Hyp; and n represents an integer of from 20 to 5,000; and wherein the polypeptide has a molecular weight at a range of from 10,000 to 500,000,000. In certain embodiments, the synthetic collagen having the structure of formula (I) has the ability to self-assemble into a triple helix to form fibrils, which allows the synthetic collagen to mimic type I collagen. It is preferred that synthetic collagen used in all the assays of the present invention have the ability to self-assemble into a triple helix to form fibrils, which allows the synthetic collagen to mimic human type I collagen.

In certain embodiments, the synthetic collagen that is used is described in U.S. Pat. No. 7,262,275. The synthetic collagen molecule was made by the method described in U.S. Pat. No. 7,262,275 (See e.g. Example 6 and Example 7). The molecular weight of the molecule was measured by the method described in the example section in the same patent as was over 1,000,000.

In certain embodiments the synthetic collagen has the following values based on GPC-MALs (gel permeation chromatography—multi-angle laser light scattering); Average molecular weight (Mₙ) 1.3×10⁴; M_{w} (weight average molecular weight)=1.6×10⁴; size average molecular weight (M_{z}) 2.0×10⁴. In other embodiments, the synthetic collagen as has the following values based on GPC-MALs (gel permeation chromatography—multi-angle laser light scattering); Average molecular weight (Mₙ) 2.8×10⁴; M_{w} (weight average molecular weight)=4.1×10⁴; size average molecular weight (M_{z}) 6.1×10⁴.

The synthetic collagen can be measured by GPC-Mals. The synthetic collagen molecules tested in the present invention were measured using the HLC-8120GPC device manufactured by Tosoh with the following conditions.
Column: TSKgel α-M (7.8 mm I.D.×30 cm)×2 (manufactured by Tosoh).
Density Detector: Differential refractometer (RI detector), polarity=(+).
MALS: DAWN HELEOS (manufactured by Wyatt Technology).
MALS Laser wave: 658 nm.
Eluent: HFIP (1,1,1,3,3,3-Hexfloro-2-propanol) manufactured by central glass+5 mM-CF₃COONa (1^{st} class manufactured by Wako Pure Chemical).
Flow Speed: 0.6 mL/min.
Column Temp.: 40° C.
RI detector Temp.: 40° C.
MALS Temp.: Room Temp.
Sample density: 2 mg/mL.
Sample amount: 100 μL.
Pre-treatment of sample: After weighing the samples, they were dissolved by adding a given amount of eluent and left at room temperature overnight. The samples gently mixed and then were then filtered through a 0.5 μm PTFE cartridge filter.

In certain embodiments n is an integer of 20 to 250. In certain embodiments n is an integer of 20 to 200. In certain embodiments n is an integer of 20 to 150. In certain embodiments n is an integer of 30 to 100. In certain embodiments n is an integer of 20 to 2,500; of 20 to 2,000; of 20 to 1,500; of 20 to 1,000; of 20 to 500; or of 20 to 250; 30 to 2,500; of 30 to 2,000; of 30 to 1,500; of 30 to 1,000; of 30 to 500; or of 30 to 250. It is preferred that the synthetic collagen molecules discussed above have the ability to self-assemble into a triple helix to form fibrils, which allows the synthetic collagen to mimic type I collagen.

Two factors to consider in choosing the synthetic collagen is solubility and ease of handling. If the molecular weight is too small, the synthetic collagen may have poor solubility characteristics. If the molecular weight is too large, the synthetic collagen may not have good handling characteristics (may be too viscous and may have poor dispersibility). Thus, a preferred synthetic collagen of the formula (I) [-(Pro-X-Gly)ₙ] has both good solubility and good handling characteristics.

The following synthetic collagen molecules were tested: n=24 (Mn=6,300); n=28 (Mw=7,500); n=49(Mn=13,000); n=60 (Mw=16,000); n=75 (Mz=20,000); n=105 (Mn=28,000); n=153 (Mw=41,000); n=229 (Mz=61,000). When testing various synthetic molecules, those having the n value from between 49-75 showed the best combination of desirable solubility and handling characteristics.

In certain embodiments of the invention, the synthetic collagen may be all one length (for example where n=49 for all synthetic molecules and in certain embodiments, the synthetic collagen may be a mixture of many different lengths (for example, but not limited to, the synthetic collagen is a mixture of molecule having n from 49-75).

### Kits

The present invention also provides a kit useful for testing platelet aggregation comprising a synthetic collagen. The synthetic collagen is as described above and can be at many different concentrations. The synthetic collagen can be supplied at a higher concentration in the vial than what would be used as the “in-test” concentration. In certain embodiments, the synthetic collagen in the vial is preferably more than 10 times the amount of the final “in-test” concentration desired. For example, the table below provides exemplary vials.

| | final conc. | working concentration. |
|---|---|---|
| | (in-test) | (concentration in vial) |
| i) | 50.0 ng/mL | 500 ng/mL |
| ii) | 25.0 ng/mL | 250 ng/mL |
| iii) | 10.0 ng/mL | 100 ng/mL |
| iv) | 5.0 ng/mL | 50 ng/mL |
| v) | 2.50 ng/mL | 25 ng/mL |
| vi) | 1.0 ng/mL | 10 ng/mL |
| vii) | 0.50 ng/mL | 5 ng/mL |

In certain embodiments the synthetic collagen is supplied in the kit at the concentration contemplated for use in the methods of the present invention to bypass the need to create dilutions of the synthetic collagen. In other words, the synthetic collagen is provided so that it is in the concentration that would be used directly in the methods of the present inventions. Thus, for example if the method called for the use of 1 mL of synthetic collagen and called for the final concentration in the assay to be 25 ng/mL, the vial in the kit would provide the synthetic collagen at a concentration of 25 ng/mL. Thus, using 1 mL out of the vial, would provide the user with a final working concentration of 25 ng/mL. As another example, if the method called for using 0.5 mL and a final concentration of 25 ng/mL, the vial in the kit would preferably contain 50 ng/mL. Thus, using 0.5 mL of this 50 ng/mL solution would give the user a final concentration of 25 ng/mL.

In certain embodiments, the synthetic collagen is provided at a concentration of 25 ng/mL to allow direct use of the collagen so that the final “in-test” collagen amount in the platelet aggregation assay would be 25 ng/mL or less. In other embodiments, the synthetic collagen is provided at an “in-test” concentration of either (for example) about 25.0 ng/mL, about 10.0 ng/mL, about 5.0 ng/mL or about 2.5 ng/mL.

In other embodiments the vial could contain a higher concentration amount and the directions included in the kit would provide instructions on the desired concentration to use in the assay to achieve the desired final concentration of synthetic collagen.

In other embodiments, the kit contains at least one single use vial and/or at least one multiple use vial of synthetic collagen. For a single use vial, the vial would contain only the amount of synthetic collagen needed for one aggregation assay. For a multiple use vial, the synthetic collagen may be supplied at the desired in-test concentration, but the vial contains more than the amount of volume needed for more than one aggregation assay. For example, if the aggregation assay called for a 1 mL solution of a final in-test concentration of synthetic collagen at 25 ng/mL, then the vial might contain 250 mL of a 25 ng/mL concentration. In this case, the user would remove 1 mL of the synthetic collagen and use it in each aggregation assay and this vial would contain enough for 250 aggregation assays. The vial could contain any amount desired for carrying out as little as one aggregation assay or many more aggregation assays as a non-limiting example 500 aggregation assays.

As non-limiting examples, in certain embodiments, the synthetic collagen is provided at concentration of 500 ng/mL to allow direct use of the collagen so that the final concentration in the aggregation assay would be 500 ng/mL or less. In other embodiments, the synthetic collagen is provided at a concentration selected within the range of about 0.500 ng/mL to about 0.050 ng/mL.

The kits of the present invention preferably contain instructions for use of the synthetic collagen in the light transmission assay using methods described herein.

In other embodiments, kits of the present invention contain more than one vial of synthetic collagen at the same concentration or in other embodiments, the kits contain more than one vial at a different concentration. Kits having more than one vial at different concentrations would be useful in the dilution profile aggregation assay of the present invention. For example, one kit of the present invention may contain vials having 8, 7, 6, 5, 4, 3 or 2 different concentrations of synthetic collagen ranging from about 0.500 ng/mL to about 0.050 ng/mL. One kit of the present invention may contain vials having 7, 6, 5, 4, or 3 different “in-test” concentrations of synthetic collagen of about 25.0 ng/mL, about 10.0 ng/mL, about 5.0 ng/mL, or about 2.5 ng/mL. Each vial would, in certain embodiments provide the synthetic collagen and the desired final “in-test” concentration and could be supplied as a single use or a multiple use vial.

In another embodiment, the kit may contain at least 5 vials each having a different concentration of synthetic collagen as follows (in ng/mL): about 50.0; about 25.0; about 10.0; about 5.0; about 2.50.

In another embodiment, the kit may contain 5 vials each having a different concentration of synthetic collagen where each vial has a concentration from within the following ranges (in ng/mL): about 50.0 to about 25.0; about 25.0 to about 10.0; about 10.0 to about 5.00; about 5.00 to about 2.50; and about 2.50 to about 1.00 ng/mL).

In another embodiment, the kit may contain 5 vials each having a different concentration of synthetic collagen where each vial has a concentration from with the following ranges: about 6.0 to about 4.0 ng/mL for the highest concentration of synthetic collagen and from about 0.3 to about 0.1 ng/mL for the lowest concentration.

The different concentrations present in the vials are chosen so that a dilution profile assay of the invention can be performed so that the donor's platelet aspirin response status can be measured against different amounts of synthetic collagen. As described above, this allows one to predict the donor's platelet aspirin response looking for a linear-like slope in response to the dilutions or in the case of a donor that is aspirin non-responsive, looking for a “bounce back” across at the dilution profile (i.e. instead of the slope corresponding to the dilutions of the synthetic collagen, there is at least one point where the slope appears to sharply rise, when it should be going down because the concentration of synthetic collagen is going down. As discussed above, this kit could be used on donors' platelets before aspirin ingestion and/or after aspirin ingestion.

It has been discovered that the nature of the vial used to store biologic or synthetic collagen can affect the collagen by activating the collagen to some degree. It is preferable that the container used to store the synthetic collage does not activate the collagen to ensure that when the synthetic collagen is removed from the vial and is introduced into a test system, the degree of activation and adherence of the synthetic collagen is due only to that test system. In other words, artifacts caused by unintentional activation by the interaction of the collagen with the container are not introduced into the aggregation assays. Collagens, including synthetic collagen, stored in generic polypropylene vials or containers are activated to an unknown degree, subsequently adhere to the container, and are thus not available to participate in the test system. The amount of collagen unavailable to the test system because it has adhered to the container and/or cap is unknown and, based on stability data, is variable. The inventors have discovered that the use of synthetic collagen that has been prepared and stored in a homopolymeric container eliminates a significant degree of variability in test results. Accordingly, it is preferred that the synthetic collagen is prepared and stored in a homopolymeric container.

Most containers that are noted as polypropylene are not a single plastic but rather are a family of plastics whose performance can be modified by including various additives during the manufacturing process. Thus, the manufacturing process itself could produce different variations of polypropylene. Further, the nature of the additives is largely unknown or disclosed to the purchaser/public as this information is considered proprietary by the manufacturers. In addition, mold release agents add another variable that could not be assessed.

It was discovered that containers that have the best long term stability and do not interact with the synthetic collagen have the following characteristics: a) the chemical structure is based on a specific, identical monomer that is repeated (a homopolymer—a polypropylene polymer consisting of identical monomer units); b) caps are made of the same material as the tubes; and c) the caps have an additional internal seal such as a silicone O ring or washer or have a secondary seal molded therein. Exemplary vials include cryovials and caps obtained from Simport (T310 Series); Lake Charles Manufacturing (54A series), and BD Falcon tubes 352096 series).

In addition, the inventors discovered that better stability was achieved when the synthetic collage was diluted with physiologic saline (with or without Thimerosal as a preservative) instead of purified water. Accordingly, kits of the present invention may contain vials of saline for dilution of synthetic collagen.

### Examples

### Example 1: Evaluation of Synthetic Collagen Using Platelet Aggregometry and Flow Cytometry Materials

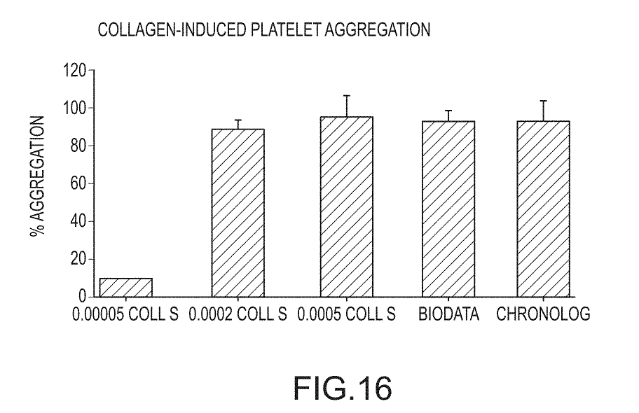

1. Collagen soluble calf skin; BioData
2. Synthetic Collagen (referred to in FIGS. 16-20 as Collagen S)
3. Collagen—type I equine; Chronolog
4. ReoPro—2.5, 5, 10 μg/mL final concentrations
5. Integrilin—1, 2, 5 μg/mL final concentrations
6. Aggrastat—1, 2, 5 μg/mL final concentrations
7. Aspisol—1, 5, 10 μg/mL final concentrations

### Methods:

### Platelet Aggregometry:

A vial of BioData calf skin collagen was reconstituted with 0.5 ml of water to make a 1.9 mg/mL solution. A vial of Synthetic collagen was reconstituted with 1 ml of Synthetic collagen diluent to make a 0.00005 mg/ml solution. Chronolog collagen was diluted with saline to make a 100 μg/ml solution. A vial of Bio/Data arachidonic acid was reconstituted with 0.5 ml of water to make a 5 mg/ml solution.

Whole blood was drawn from healthy individuals before and after ingestion of a 325 mg aspirin tablet into sodium citrate (3.2%) and centrifuged to make PRP and PPP. The PAP-8E aggregometer was blanked with 250 μl of PPP. 200 μl of PRP was pipetted into each of 4 stirbar-containing cuvettes. 25 μl of anti-platelet agent was added to the PRP. After a 3 minute incubation period, 25 μl of saline or agonist was added to the cuvettes. The aggregation response was monitored until maximum aggregation was been achieved.

### Flow Cytometry:

A vial of Bio/Data calf skin collagen was reconstituted with 0.5 mL of water to make a 1.9 mg/mL solution, A vial of Synthetic collagen was reconstituted with 1 ml of Synthetic collagen diluent to make a 0.0005 mg/mL solution. Chronolog collagen was diluted with saline to make a 100 μg/mL solution. A stock 2% paraformaldehyde solution was diluted with calcium-free Tyrode's buffer to make a 1% paraformaldehyde solution. A set of tubes containing 1 mL of 1% paraformaldehyde was prepared. A second set of tubes which contained 30 μl of collagen reagent and 30 μl of anti-platelet drug was prepared and set in a 37° C. heating block Whole blood was drawn from healthy individuals into sodium citrate. 240 μl of citrated blood was added to the tubes at 15-20 second intervals and gently mixed. After a 3 minute incubation period, 50 μl of activated blood was transferred to the corresponding paraformaldehyde-containing tube. After a 30 minute incubation at 4° C., the samples were centrifuged at 1,600 rpm for 10 minutes and the supernatant was removed. The cell pellet was resuspended in 750 μl of Tyrode's buffer. 10 μl each of CD61FITC and CD62PE (BD Biosciences) was added to a set of clean tubes. 100 μl of resuspended cells was added to the antibody tubes. After a 30 minute incubation period in the dark at room temperature, 700 μl of Tyrode's buffer was added to each tube and the samples were analyzed on the flow cytometer (EPICS-XL, Beckman-Coulter). Platelet activation was assessed in terms of the percentage of platelets expressing P-selectin and the percentage of aggregated platelets.

### Results:

Multiple concentrations lots of Synthetic collagen were tested using light transmittance aggregometry. The aggregation response to Synthetic collagen was compared to that of collagen reagents from Bio/Data and Chronolog. (See FIG. 16) While 0.00005 mg/mL Synthetic collagen produced minimal aggregation, the higher concentrations (0.0002 and 0.0005 mg/mL) produced a comparable level of aggregation to that of the other collagen reagents.

The response of aspirinized platelets to the various collagen reagents was slightly attenuated compared to the response of non-aspirinized platelets. The extent of aggregation was 9% (Bio/Data) to 20% (Synthetic collagen) lower with aspirinized platelets compared to non-aspirinized platelets.

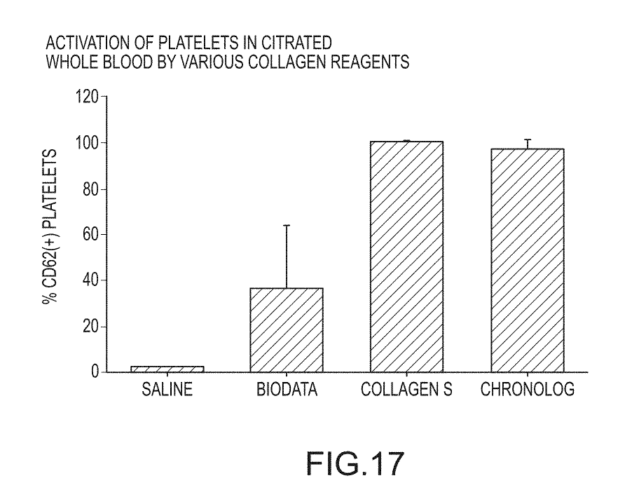

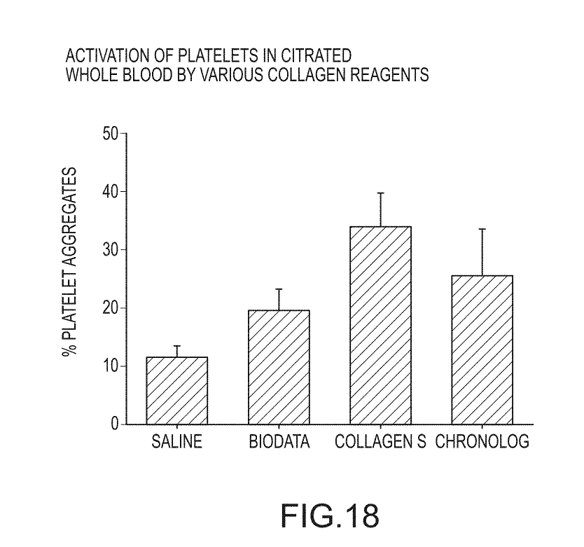

The ability of the various collagen reagents to induce platelet activation was assessed using whole blood flow cytometry. Platelet activation was assessed in terms of two parameters: P-selectin expression and formation of platelet aggregates. In this assay, platelet aggregates are defined as CD61(+) events with a size (forward angle light scatter) greater than that of the unaggregated platelet population. All collagen reagents were able to induce P-selectin expression on the platelet surface (FIG. 17) although the BioData collagen was much less effective compared to the other reagents. A similar trend was observed with the formation of platelet aggregates in whole blood (FIG. 18).

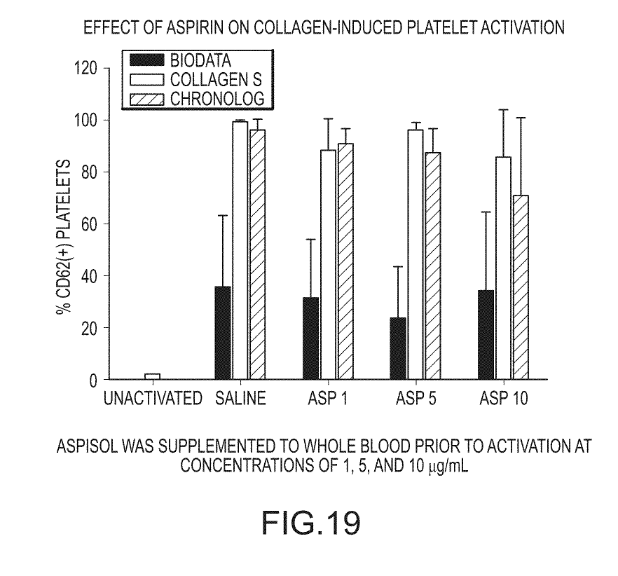

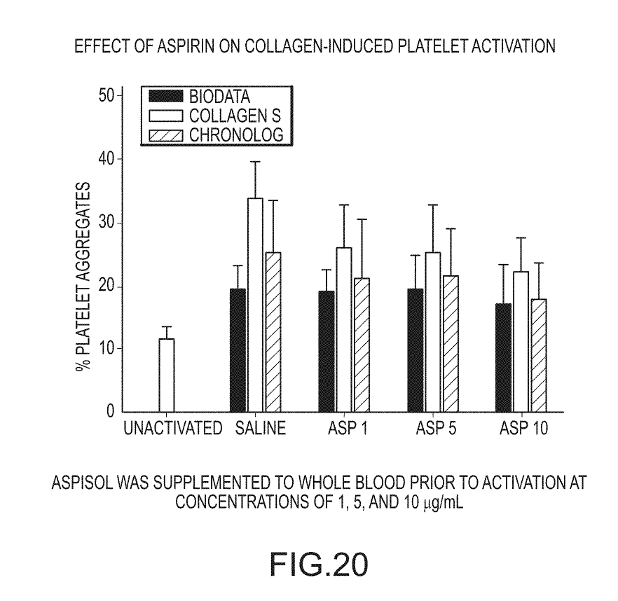

For the flow cytometry studies, a soluble form of aspirin, Aspisol®, was supplemented to citrated whole blood prior to activation. Aspisol had little effect on collagen-induced P-selectin expression, but had a concentration-dependent effect on the formation of platelet aggregates. This effect was most readily seen with the Synthetic collagen reagent (See FIGS. 19 and 20).

1) A platelet aggregation assay for determining a donor's aspirin sensitivity status and compliance, the assay comprising the use of synthetic collagen at a final concentration in the assay from about 0.050 ng/mL to about 500 ng/mL.

2) A method for determining a donor's aspirin sensitivity status, the method comprising performing a platelet aggregation assay by:
a) combining a first platelet rich sample obtained from the donor with an amount of synthetic collagen less than about 500 ng/mL to form a first treated sample, wherein the donor has not ingested aspirin for a time period of least about 24 hours;
b) measuring aggregation of the first treated sample to obtain a first readout, wherein the first readout determines the donor's baseline level in the absence of ingested aspirin;
c) combining a second platelet rich sample obtained from the donor after the donor has ingested aspirin with an amount of synthetic collagen less than about 500 ng/mL to form a second treated sample;
d) measuring platelet aggregation the second treated sample to obtain a second readout;
e) comparing the baseline level in the absence of ingested aspirin with the second treated sample readout, wherein the comparison determines the donor's aspirin sensitivity status.

3) The method of claim 2 wherein in the second platelet rich sample aspirinated instead of having the donor ingest the aspirin.

4) The method of claim 1 wherein the amount of synthetic collagen less than about 50.0 ng/mL.

5) The method of claim 1 wherein the amount of synthetic collagen less than about 25.0 ng/mL.

6) The method of claim 1 wherein the amount of synthetic collagen less than about 10.0 ng/mL.

7) The method of claim 1 wherein the amount of synthetic collagen less than about 1.0 ng/mL.

8) The method of claim 1 wherein the amount of synthetic collagen less than about 0.50 ng/mL.

9) The method of claim 1 wherein the amount of synthetic collagen less than about 0.05 ng/mL.

10) The method of claim 2 further comprising performing a dilution profile analysis to further analyze the donor's aspirin reactivity status, the method comprising:
f) mixing multiple different platelet rich samples obtained from the donor before ingesting aspirin, each mixed independently with a different synthetic collagen dilution amount over a range of concentrations, to obtain multiple different treated baseline samples to obtain a baseline dilution profile over the range of different concentrations;
g) measuring platelet aggregation through the multiple different treated baseline samples to obtain a baseline dilution profile readout;
h) mixing multiple different platelet rich samples obtained from the donor after ingesting aspirin, each mixed independently with a different synthetic collagen dilution amount over a range of concentrations, to obtain multiple different treated post aspirin samples to obtain a post aspirin dilution profile over the range of different concentrations;
wherein the same dilution amounts are used for the baseline dilution profile in step f) and the post aspirin dilution profile in step h);
i) measuring platelet aggregation through the multiple different treated post aspirin samples to obtain a dilution profile post aspirin readout;
j) analyzing the dilution profile post aspirin readout against the baseline dilution profile readout to determine the level of the donor's aspirin sensitivity.

11) A method for determining a donor's aspirin sensitivity status, the method comprising performing a dilution profile analysis using a platelet aggregation assay by:
a) mixing multiple different platelet rich samples obtained from the donor before ingesting aspirin, each mixed independently with a different synthetic collagen dilution amount over a range of concentrations, to obtain multiple different treated baseline samples to obtain a baseline dilution profile over the range of different concentrations;
b) measuring platelet aggregation through the multiple different treated baseline samples to obtain a baseline dilution profile readout;
c) mixing multiple different platelet rich samples obtained from the donor after ingesting aspirin, each mixed independently with a different synthetic collagen dilution amount over a range of concentrations, to obtain multiple different treated post aspirin samples to obtain a post aspirin dilution profile over the range of different concentrations;
wherein the same dilution amounts are used for the baseline dilution profile in step a) and the post aspirin profile in step c);
d) measuring platelet aggregation through the multiple different treated post aspirin samples to obtain a dilution profile post aspirin readout;
e) comparing the dilution profile post aspirin readout against the baseline dilution profile readout to determine the level of the donor's aspirin sensitivity.

12) The method of claim 11 where instead of having the donor ingest the aspirin, the platelet rich samples are aspirinated.

13) A method for predicting a donor's aspirin sensitivity status, the method comprising performing a dilution profile analysis using a platelet aggregation assay by:
a) mixing multiple different platelet rich samples obtained from the donor, each mixed independently with a different synthetic collagen dilution amount over a range of concentrations, to obtain multiple different samples to obtain a dilution profile over the range of different concentrations;
b) measuring platelet aggregation through the multiple different treated samples to obtain a dilution profile readout;
c) analyzing the dilution profile readout to predict the donor's aspirin sensitivity status.

14) The method of claim 13 wherein the platelet aggregation assay utilizes a light transmission assay.

15) The method of claim 13 wherein the platelet aggregation assay utilizes a flow cytometer.

16) The method of claim 14 wherein the readout is the primary aggregation, primary slope, area under the curve, or a combination thereof.

17) The method of claim 16 wherein when the readout is primary aggregation, and wherein when the baseline level in the absence of ingested aspirin shows platelet aggregation and when the second treated sample does not shows a significant reduction in platelet aggregation as compared to the level of platelet aggregation in the baseline level, the donor is determined to be aspirin non-responsive.

18) The method of claim 16 wherein when the readout is primary aggregation, and wherein when the baseline level in the absence of ingested aspirin shows platelet aggregation and when the second treated sample does not shows a significant reduction in platelet aggregation as compared to the level of platelet aggregation in the baseline level, the donor is determined to be aspirin non compliant.

19) The method of claim 3 wherein when the readout is primary aggregation, and wherein when the baseline level in the absence of ingested aspirin shows platelet aggregation and when the second treated sample shows a significant reduction in platelet aggregation as compared to the level of platelet aggregation in the baseline level, the donor is determined to have average aspirin sensitivity.

20) The method of claim 13 wherein the multiple different synthetic collagen amounts include 3, 4 or 5 different synthetic collagen amounts within the sensitive region (SR);
wherein the sensitive region (SR) is the range of synthetic collagen concentrations in which measured platelet activity/aggregation is reduced with decreasing collagen concentrations in an average donor with an average aspirin sensitivity.

21) The method of claim 20 wherein the different synthetic collagen dilution amounts comprise 5 different synthetic collagen amounts chosen from within the concentration range of about 50.0 ng/mL to about 0.050 ng/mL.

22) The method of claim 20 wherein the 5 different synthetic collagen amounts include: 50.0 ng/mL; 25.0 ng/mL; 10.0 ng/mL; 5.0 ng/mL; and 2.50 ng/mL.

23) The method of claim 20 wherein the 5 different synthetic collagen amounts include one concentration from within each of the following ranges: 50.0-25.0 ng/mL; 25.0-10.0 ng/mL; 10.0-5.00 ng/mL; 5.00-2.50 ng/mL; and 2.50-1.00 ng/mL).

24) The method of claim 20 wherein the different synthetic collagen dilution amounts comprise 3 different synthetic collagen amounts chosen from within the concentration range of about 50.0-25 ng/mL for the highest concentration of synthetic collagen and 2.50-0.50 ng/mL for the lowest concentration.

25) The method of claim 16 wherein the donor is determined to be aspirin resistant, wherein when instead of showing a linear-like reduction of the platelet aggregation, slope or area under the curve values correlating to the reduction in the concentration of synthetic collagen, there is an increase in platelet aggregation, slope or area under the curve values in at least one concentration of synthetic collagen when there should be a corresponding decrease in platelet aggregation, slope or area under the curve values.

26) The method of claim 17 wherein the donor's platelet aspirin response status is selected from the group consisting of aspirin hypersensitive, average aspirin sensitive, and aspirin non-responsive.

27) The method of claim 1 wherein the synthetic collagen comprises a polypeptide having a peptide fragment represented by the formula (I)
-(Pro-X-Gly)ₙ (I)
wherein X represents Hyp; and n represents an integer of from 20 to 250.

28) A kit for testing platelet aggregation platelet aggregation assay, comprising:
a) a vial of synthetic collagen at a concentration of from about 0.50 ng/mL to about 500 ng/mL; wherein the synthetic collagen comprises a polypeptide having a peptide fragment represented by the formula (I)
-(Pro-X-Gly)ₙ (I)
wherein X represents Hyp; and n represents an integer of from 20 to 250; and
b) instructions for use of the synthetic collagen in the platelet aggregation assay; and wherein the vial is a homopolymer of polypropylene.

29) The kit of claim 27 further comprising multiple additional vials of synthetic collagen at different concentrations ranging from about 0.50 ng/mL to about 500.0 ng/mL.

30) A platelet aggregation assay for determining a donor's aspirin therapy compliance, the assay comprising the use of synthetic collagen at a final concentration in the assay from about 0.050 ng/mL to about 640 ng/mL.

31) A method for determining a donor's aspirin therapy compliance, the method comprising performing a platelet aggregation assay by:
a) combining a first platelet rich sample obtained from the donor with an amount of synthetic collagen less than about 500 to 640 ng/mL to form a first treated sample, after the donor has ingested aspirin;
b) measuring aggregation of the first treated sample to obtain a first readout, wherein the first readout determines the donor's baseline level in the presence of ingested aspirin;
c) combining a second platelet rich sample obtained from the donor after the donor has been on an aspirin therapy regimen with an amount of synthetic collagen less than about 500 to 640 ng/mL to form a second treated sample;
d) measuring platelet aggregation of the second treated sample to obtain a second readout;
e) comparing the baseline level with the second treated sample readout to ascertain whether the donor has complied with the aspirin therapy based on whether platelet aggregation levels in the second readout are similar to the base line levels, wherein the comparison determines the donor's aspirin therapy compliance.

32) The method of claim 31 wherein the assessment of platelet aggregation is through light transmission assay or flow cytometry.

33) The method of claim 32 further comprising obtaining a third platelet rich sample to which a solution of aspirin is added, and assessing platelet aggregation on the third platelet rich sample.

34) The method of claim 33 wherein the amount of synthetic collagen less than about 100 ng/mL.

35) The method of claim 33 wherein the amount of synthetic collagen less than about 50.0 ng/mL.

36) The method of claim 33 wherein the amount of synthetic collagen less than about 25.0 ng/mL.

37) The method of claim 30 wherein the synthetic collagen comprises a polypeptide having a peptide fragment represented by the formula (I)
-(Pro-X-Gly)ₙ (I)
wherein X represents Hyp; and n represents an integer of from 20 to 250.
